(19) United States
(12) Patent Application Publication
(10) Pub. No.: US 2019/0353646 A1
(43) Pub. Date: Nov. 21, 2019

(54) DEVICE AND METHOD TO DETERMINE OR QUANTIFY THE PRESENCE OF AN ANALYTE MOLECULE

(71) Applicants: Luka FAJS, Singapore (SG); Robert S. MARKS, Singapore (SG); Evgeni ELTZOV, Singapore (SG)

(72) Inventors: Luka FAJS, Singapore (SG); Robert S. MARKS, Singapore (SG); Evgeni ELTZOV, Singapore (SG)

(21) Appl. No.: 16/463,394

(22) PCT Filed: Nov. 23, 2016
(86) PCT No.: PCT/SG2016/050578

§ 371 Date: May 23, 2019

(51) Int. Cl. G01N 33/52; G01N 33/569; G01N 33/543

(52) U.S. Cl. CPC G01N 33/525; G01N 33/569; G01N 33/54386; G01N 2333/255; G01N 2333/075; G01N 2333/11; G01N 2333/245; G01N 2333/185

(57) ABSTRACT

This disclosure relates to a device to determine or quantify the presence of an analyte molecule, virus or cell of interest in a sample. The present disclosure also relates to a method to determine or quantify the presence of an analyte molecule, virus or cell of interest in a sample, a method of preparing the device of the disclosure, the use of the device of the disclosure for determining or quantifying the presence of an analyte molecule, virus or cell of interest in a sample and a kit of parts comprising the device of the disclosure.

### CROSS-REFERENCE TO RELATED APPLICATIONS

This application is a national stage entry according to 35 U.S.C. § 371 of PCT Application No PCT/SG2016/050578 filed on Nov. 23, 2016, which is incorporated by reference herein in its entirety.

### FIELD OF THE DISCLOSURE

This disclosure relates to a device to determine or quantify the presence of an analyte molecule, virus or cell of interest in a sample. The present disclosure also relates to a method to determine or quantify the presence of an analyte molecule, virus or cell of interest in a sample, a method of preparing the device of the disclosure, the use of the device of the disclosure for determining or quantifying the presence of an analyte molecule, virus or cell of interest in a sample and a kit of parts comprising the device of the disclosure.

### BACKGROUND

A number of classical methods exist to identify pathogens, such as cell plate culture, immunoassays, and nucleic acid related tests. However, depending on the method, any one of them may suffer from one or more disadvantages such as low sensitivity, high price, assay complexity, requirement of a lab environment, and more. In addition, none of these methods can be developed for domestic or field consumers in the near-to midterm future. Two types of devices have reached the necessary requirements to make them consumer friendly. One is the biosensor-based glucometer, and the other is the pregnancy test. There are technologies in development seeking to combine these successful technologies.¹ However, presently the gold standard low-cost rapid test is the lateral flow immunoassay (LFA).² By 2010, more than 100 companies worldwide had produced a wide range of LFA tests with a total market valued at over 3.36 billion USD.³ Different parameters (e.g., cost efficiency, portability, simplicity, and speed) made LFA more attractive than other conventional detection approaches [e.g., enzyme-linked immunosorbent assay (ELISA), polymerase chain reaction (PCR), cell culture]. The format of LFA uses the same rationale as ELISA, where immobilized antibody or antigen is bound onto nitrocellulose membrane instead of a plastic well. The membrane enables a one-step assay, a major advantage when compared to ELISA. Typical LFA is based on four segments, a sample pad, a conjugate pad, a nitrocellulose membrane, and an absorbent pad, each serving a given purpose, overlapping one another, and combined on a plastic backing support. The sample, added onto the sample pad, migrates through the membrane and is captured by the antibodies immobilized within the nitrocellulose membrane to produce a visible and putatively measurable colorimetric signal. There are various LFA formats, biorecognition molecules, labels, detection systems, and applications.⁴⁻⁷ LFAs have been used to monitor infectious agents,^{5,8,9} nucleic acids,^{10,11} proteins,¹² cells,¹³ veterinary drugs,^{14,15} toxins,¹⁶⁻¹⁸ and pesticides.¹⁹ The most popular test to date is the well-known and well-received pregnancy test. The biggest disadvantage of LFA is that virtually all tests are qualitative, or sometimes semiquantitative, and despite the use of signal amplification (enzyme labels), insufficient sensitivity is obtained.^{12,20-22} In the case of the popular pregnancy test, there is sufficient amount of target metabolite, and therefore, there is no need for quantification, as a positive presence of hormone is sufficient to provide the sought-after answer. In other cases (e.g., viral load in HIV treatment or severe Dengue triage), quantification would be needed.

There has been new advance in developing cost-effective and rapid bacterial testing based on the lateral flow platform. Lateral flow sensors for Escherichia coli²³, Listeria²⁴, Salmonella²⁵ and Streptococcus²⁶ were developed. However, low sensitivity to the target microorganisms (10⁵ to 10⁶ cfu/mL) and the usually reduced specificity continue to pose problems. ELISA based technologies (e.g. chemiluminescence²⁷, electrochemistry²⁸, colorimetry 29,30,31 provide more sensitive results, however they are more complicated to run and are time consuming.

There is a need in the art to develop devices and methods overcoming the above mentioned disadvantages of LFAs and ELISA based detection approaches.

### SUMMARY

It is an object of the present disclosure to meet the above need by providing devices comprising or consisting of a unit of stacked layers as described herein. This structure of layers allows the liquid to migrate from the lower to the upper layers or from the upper layer to the lower layers. The order of the membranes is, from the lowest to the uppermost, the sample layer, conjugation layer, blocking layer and absorption layer. Similar to lateral flow setups, the liquid with the putative target analyte is added to the sample pads, and thereafter it migrates upwards to the conjugation layer. It then conjugates with anti-analyte antibodies (laying in wait) and then further migrates as analyte/antibody complex to the capture layer. This technology is different from prior art devices by its modified nitrocellulose layers and their structure, which are selective to the target analyte molecules. This technology allows for a simple, semi-quantitative, fast (less than 5 min) and portable measurement of the target analyte molecule. Moreover, this is a generic technology easily adapted towards any target analyte in the future. In addition, the key usefulness of this technology is that it enables multiplex devices.

In a first aspect, the present disclosure is thus directed to a device to determine or quantify the presence of an analyte molecule, virus or cell of interest in a sample, wherein said device consists of or comprises at least one unit of stacked layers comprising: at least one blocking layer comprising a membrane and a plurality of said analyte molecules, virus or cells of interest attached to the membrane.

In a second aspect, the present disclosure is directed to a method to prepare the device of the disclosure, comprising: incubating and drying the membrane of the at least one conjugation layer and a sample comprising the first binding molecule; immobilizing, e.g., covalently immobilizing, the plurality of analyte molecules, virus or cells of interest on the membrane of the at least one blocking layer; and stacking the layers of the unit of stacked layers to form the device of the disclosure.

In a further aspect, the disclosurerelates to the use of the device of the disclosure to determine or quantify the presence of an analyte molecule, virus or cell of interest in a sample.

Further, the present disclosure is directed to a method to determine or quantify the presence of an analyte molecule, virus or cell of interest in a sample, comprising: contacting the sample and the device of the disclosure; and determining or quantifying the presence of the analyte molecule by detecting a reporter molecule dependent signal.

Finally, the present disclosure relates in a fifth aspect to a kit of parts comprising at least one device of the disclosure.

### BRIEF DESCRIPTION OF THE DRAWINGS

The disclosure will be better understood with reference to the detailed description when considered in conjunction with the non-limiting examples and the accompanying drawings.

FIG. 1 shows a schematic presentation of the flow pad biosensor/device of the disclosure. In general it consists of different nitrocellulose pads with various components: (1) sample pad (empty) where the analyte sample will be placed; (2) pad with anti-analyte bioreporter molecule linked to some marker; (3) blocker pad, with immobilized analyte; (4) measuring pad (depends on the marker in pad 2 or empty or with specific substrate). There are two main possibilities that can happen during measurements. In the first, sample with target analyte, after deposition under the pad 1, it will migrate to pad 2, where it will be connected to bioreporter molecules (in this case antibodies). The complex will then migrate through pad 3 and reach pad 4 to produce a measured signal since the complex is already formed. Samples without target analyte will migrate from pad 1 to 2 and then will move antibodies (in this case) to the blocking pad 3 where they will be captured to the immobilized analyte and stopped from migrating to the next pad. Thus, no visible signal will be observed.

FIG. 2 shows determination of optimal operating conditions of the stacks sensor. A. Required minimum sample volume to reach the uppermost layer and B. Effect of the number of blocking layers to reduce false responses of the system to clear water. C. Proof of concept of the optimized immunoassay to detect target bacteria in a water sample.

FIG. 3 shows the effect of drying different volumes of the reporters (Gold nano particles) on the various pads.

FIG. 4 shows the number of blocking layers required to reduce a false positive response by the assay.

FIG. 5 shows the effect of the blocking pad on the flow of the reporter molecule to the sensor layers where the enzymatic reaction occurs.

FIG. 6 shows the effect of the HRP (horseradish peroxidase) concentration of the bioluminescent responses.

FIG. 7 shows optimization of the immobilization procedures.

FIG. 8 shows determination of leaching of the antibodies from the nitrocellulose membrane.

FIG. 9 shows Response of the StackPad system (device of the disclosure). A. live pads image and B. JPEG signals analyzing based on ImageJ software and C. colorimetric ELISA to different E. coli DH5 strain concentrations.

FIG. 10 shows specificity of the two used approaches: (A) ELISA; (B) stack pad immunoassay to the anti-E. coli antibodies.

FIG. 11 shows the effect of the blocking layer on the stack pad assay functionality.

FIG. 12 shows the effect of the blocking layer numbers and antibodies concentrations on the sensor false response generation.

FIG. 13 shows the correlation curve of the (A) ELISA and (B) stack pad assay to the different DH5α concentrations.

FIG. 14 shows the specificity of the StackPad assay to different bacterial strains. A. live pads image and B. JPEG signals analyzing based on ImageJ software.

FIG. 15 shows response of the StackPad system to different environmental water. In addition, the system was exposed to DH5-α cells (10³ cfu/mL) as positive spiked samples and clear water as negative controls.

FIG. 16 shows response of the flow pad biosensor on electrochemical approaches.

FIG. 17 shows the addition of a stopping layer after the blocking area.

FIG. 18 shows the effect of covalent binding approach of the attachment of biological molecule on fiberglass paper.

FIG. 19 shows the effect of the immobilization approach on the efficiency of immobilization biological molecules above PVDF membrane.

FIG. 20 shows the StackPad structure and experiments parameters for determination protein G in the water samples using immobilized Neisseria gonorrhoeae cells.

FIG. 21 shows the determination Neisseria gonorrhoeae with StackPad setup.

FIG. 22 shows the determination Dengue virus (as NS1 antigen) with StackPad setup.

FIG. 23 shows examples of the multiplex aspects of the device of the disclosure.

FIG. 24 shows the fixing of the layers of the unit of stacked layers by two alternative approaches.

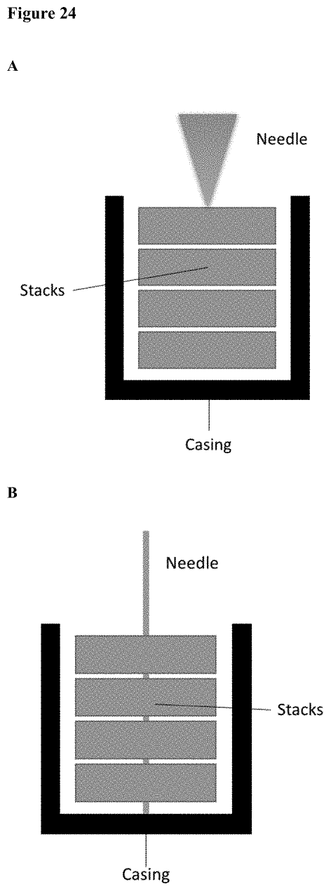

### DETAILED DESCRIPTION

The present inventors surprisingly found that the specific modification of membranes and their specific structure as different layers in a unit of stacked layers as described herein allows the preparation of a portable detection device and the fast and cheap detection or quantitation of analyte molecules.

Therefore, in a first aspect, the present disclosure is thus directed to a device to determine or quantify the presence of an analyte molecule, virus or cell of interest in a sample, wherein said device consists of or comprises at least one unit of stacked layers comprising: at least one blocking layer comprising a membrane and a plurality of said analyte molecules, virus or cells of interest attached to the membrane.

In some aspects, the unit of stacked layers further comprises:
sample layer comprising a membrane;
at least one conjugation layer comprising a membrane and a first binding molecule binding to the analyte molecule, virus or cells of interest, wherein
(a) the first binding molecule is conjugated to a reporter molecule; or
(b) a second conjugation layer comprises a membrane and a second binding molecule binding to the first binding molecule, wherein the second binding molecule is conjugated to a reporter molecule; and
   absorption layer comprising a membrane.

The term “device”, as used herein, relates to a tool or sensor that comprises or consists of a unit of stacked layers as described herein and allows the determination and/or quantitation of analyte molecules, virus or cell of interest in a sample.

“Determine”, as used herein, generally refers to the analysis of a species (such as one or more analyte molecules), for example, quantitatively or qualitatively, and/or the detection of the presence or absence of the species. The term “detect” in the present disclosure means both a determination of the quantity of an analyte molecule and the presence of an analyte molecule. It is to be understood that the term “quantify” as used herein refers to both quantitative measurement and qualitative measurement of a molecule in a sample. Said terms are used in the broadest sense to include both qualitative and quantitative measurements of a specific molecule, herein measurements of a specific analyte molecule such as a protein or cell. In one aspect, a detection method as described herein is used to identify the mere presence of an analyte molecule of interest in a sample. In another aspect, the method can be used to quantify an amount of analyte molecule in a sample. In still another aspect, the method can be used to determine the relative binding affinity of an analyte molecule of interest for a target molecule.

“Analyte” and “analyte molecule,” as used herein, refer to a molecule that is analyzed by the device and methods of the disclosure, and includes, but is not limited to, small molecules, polypeptides (proteins), polypeptide fragments, antibodies, antibody fragments, (bacterial) cells, virus particles (virions), natural ligands, DNA, RNA, nucleotide primers and the like. In the device and methods of the disclosure, an analyte molecule has a binding affinity for a binding molecule.

The term “sample,” as used herein, is used in its broadest sense. A “biological sample,” as used herein, includes, but is not limited to, any quantity of a substance (analyte molecule) from a living thing or formerly living thing that can be solubilized in a first extraction buffer optionally containing a surfactant or detergent. Such living things include, but are not limited to, mammals, humans, non-human primates, mice, rats, monkeys, dogs, rabbits, and other animals; plants; single celled organisms such as yeast and bacteria and viruses. Such substances include, but are not limited to, blood, (e.g., whole blood), plasma, serum, urine, amniotic fluid, synovial fluid, endothelial cells, leukocytes, monocytes, other cells, organs, tissues, bone marrow, lymph nodes, and spleen, e.g., from resected tissue or biopsy samples; and cells collected, e.g. by centrifugation, from any bodily fluids; and primary and immortalized cells and cell lines. Samples can include fresh samples and historical samples. However, the term “sample” also includes environmental samples, such as water samples or smear tests of diverse items. As used herein, a “cell based sample” is understood as a sample wherein substantially all (e.g. at least 90%, at least 95%, at least 98%, at least 99%) of the analyte molecule present in the sample for detection is present inside cells of the sample (i.e., not in serum, extracellular fluid, cell culture media). In some aspects, the sample is a liquid sample.

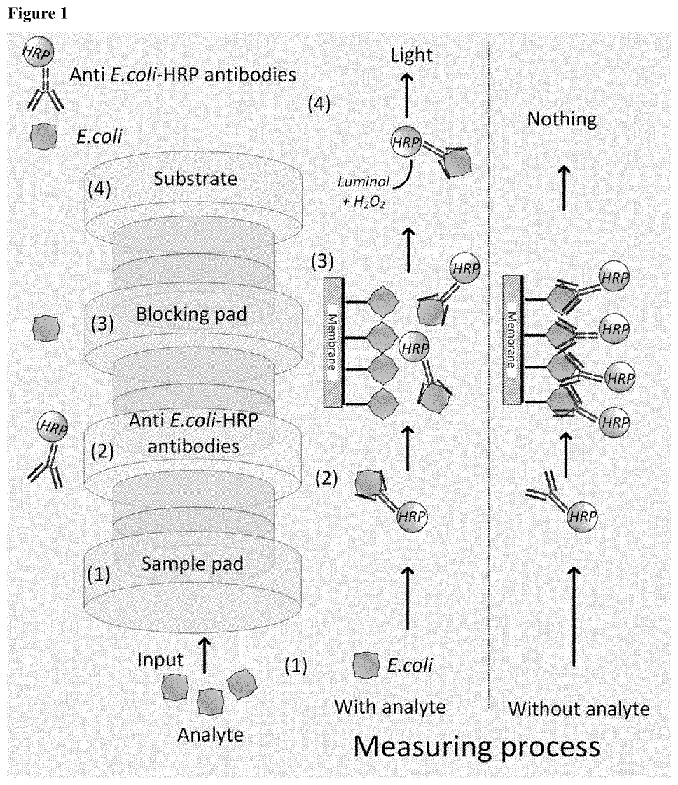

The term “unit of stacked layers”, as used herein, refers to a unit comprising a sample layer, at least one conjugation layer, at least one blocking layer and an absorption layer as defined herein. The unit of stacked layer may further comprise one or more separation layer and/or a stopping layer as defined herein. Each of said layers has a spatial form wherein its length and width are bigger than its height. In various aspects, the length and width have approximately the same size. Thus, the resulting form of the layer is a square. In various other aspects of the disclosure, the length and/or width of the layer are at least 2-fold, at least 5-fold, at least 10-fold, at least 20-fold, at least 25-fold, at least 50-fold or at least 100-fold bigger than the height. The areas of the layers providing the largest surface are contacted with each other and therefore they are stacked. The outer layers of such a stacked unit are usually the sample layer and the absorption layer. An example of a unit of stacked layers of the present disclosure is shown in FIG. 1.

As used herein, the term “membrane” refers to a natural or synthetic/artificial membrane. The terms “synthetic membrane” or “artificial membrane” refer to a man-made membrane that is produced from organic material, such as polymers and liquids, as well as inorganic materials. A wide variety of synthetic membranes are well known in the art. In various aspects, the membranes of the sample layer, the at least one conjugation layer, the at least one blocking layer and the absorption layer are independently selected from the group consisting of cellulose acetate membrane, nitrocellulose membrane, cellulose ester membrane, polysulfone (PS) membrane, polyether sulfone (PES) membrane, polyacrilonitrile (PAN) membrane, polyamide membrane, polyimide membrane, polyethylene and polypropylene (PE and PP) membrane, polytetrafluoroethylene (PTFE) membrane, polyvinylidene fluoride (PVDF) membrane, polyvinylchloride (PVC) membrane and fiberglass paper membrane.

In other various aspects, the membrane is a porous membrane. As used herein, the term “porous membrane” refers to a membrane having a plurality of pores or throughbores that permit gas or vapour molecules to pass across the membrane.

According to one aspect of the device of the present disclosure, the membrane is a porous membrane having a nominal pore size in a range of 0.01 μm to 30 μm, e.g., between 0.2 μm to 5 μm. According to one aspectment of the present disclosure, the membrane has a nominal pore size in a range of 0.02 μm to 1 μm, e.g., 0.35 μm to 0.8 μm.

According to one aspect of the membrane of the present disclosure, the original, un-coated porous substrate is a porous membrane having a porosity of 0.40 to 0.99, e.g., 0.70 to 0.90. According to one aspect of the present disclosure, the membrane has a porosity of 0.40 to 0.99, e.g., 0.60 to 0.90. “Porosity”, as used herein, refers to the volume of the pores divided by the total volume of the porous substrate.

The term “binding molecule” as used herein includes molecules that contain at least one binding site that specifically binds to the analyte molecule or to the first binding molecule. By “specifically binds” it is meant that the binding molecules exhibit essentially background binding to the analyte molecule or to the first binding molecule. The term “specificity”, as used herein, refers to the ability of a binding moiety to bind preferentially to one analyte molecule, versus a different antigen, and does not necessarily imply high affinity (as defined further herein). A binding moiety that can specifically bind to and/or that has affinity for a specific analyte molecule is said to be “against” or “directed against” said antigen or antigenic determinant. A binding molecule according to the disclosure is said to be “cross-reactive” for two different analyte molecules if it is specific for both these different analyte molecules. The term “affinity”, as used herein, refers to the degree to which a binding molecule binds to an analyte molecule so as to shift the equilibrium of free analyte molecule and binding molecule toward the presence of a complex formed by their binding. Thus, for example, where an analyte molecule and binding molecule are combined in relatively equal concentration, a binding molecule of high affinity will bind to the available analyte molecule so as to shift the equilibrium toward high concentration of the resulting complex. The dissociation constant (K_{d}) is commonly used to describe the affinity between the binding molecule and the its target. Typically, the dissociation constant is lower than 10⁵ M. For example, the dissociation constant is lower than 10⁶ M, e.g., lower than 10⁷ M, e.g., the dissociation constant is lower than 10⁸ M.

The terms “specifically bind” and “specific binding”, as used herein, generally refers to the ability of a binding domain to preferentially bind to a particular analyte molecule that is present in a homogeneous mixture of different molecules. In certain aspects, a specific binding interaction will discriminate between desirable and undesirable molecules in a sample, in some aspects more than about 10 to 100-fold or more (e.g., more than about 1000- or 10,000-fold).

In various aspects, the first and the second binding molecules are independently selected from the group consisting of protein, e.g., an antibody, nucleotide and natural ligand. The term “protein”, as used herein, relates to one or more associated polypeptides, wherein the polypeptides consist of amino acids coupled by peptide (amide) bonds. The term polypeptide refers to a polymeric compound comprised of covalently linked amino acid residues. The amino acids are, for example, the 20 naturally occurring amino acids glycine, alanine, valine, leucine, isoleucine, phenylalanine, cysteine, methionine, proline, serine, threonine, glutamine, asparagine, aspartic acid, glutamic acid, histidine, lysine, arginine, tyrosine and tryptophan.

As used herein, the term “antibody” refers to an intact immunoglobulin including monoclonal antibodies, such as chimeric, humanized or human monoclonal antibodies, or to an antigen-binding and/or variable domain comprising fragment of an immunoglobulin that competes with the intact immunoglobulin for specific binding to the binding partner of the immunoglobulin, e.g., CD1a. Regardless of structure, the antigen-binding fragment binds with the same antigen that is recognized by the intact immunoglobulin. The term “antibody” as used herein includes immunoglobulins from classes and subclasses of intact antibodies. These include IgA, IgD, IgE, IgG, and IgM, and several of these may be further divided into subclasses (isotypes), e.g., IgA1, IgA2, IgG1, IgG2, IgG3 and IgG4 as well as antigen-binding fragments thereof.

Antigen-binding fragments include, inter alia, Fab, F(ab′), F(ab′)2, Fv, dAb, Fd, complementarity determining region (CDR) fragments, single-chain antibodies (scFv), bivalent single-chain antibodies, diabodies, triabodies, tetrabodies, (poly)peptides that contain at least a fragment of an immunoglobulin that is sufficient to confer specific antigen binding to the (poly)peptide, etc. The above fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, edited by E. Harlow and D. Lane (1988), Cold Spring Harbor Laboratory, Cold Spring Harbor, N.Y., which is incorporated herein by reference. A binding molecule or antigen-binding fragment thereof may have one or more binding sites. If there is more than one binding site, the binding sites may be identical to one another or they may be different.

The term “nucleotide” as used herein refers to ribonucleotides, deoxyribonucleotides, dideoxynucleotides, acyclic derivatives of nucleotides, and functional equivalents thereof, of any phosphorylation state. Functional equivalents of nucleotides are those that may be functionally substituted for any of the standard ribonucleotides or deoxyribonucleotides in a polymerase or other enzymatic reaction as, for example, in an amplification or primer extension method. Functional equivalents of nucleotides are also those that may be formed into a polynucleotide that retains the ability to hybridize in a sequence specific manner to a target polynucleotide.

The term “ligand” as used herein refers to a molecule or more generally to a compound which is capable of binding to a target protein. A target protein may have a co-factor or physiological substrate bound thereto. The ligand of interest may bind elsewhere on the protein or may compete for binding e.g. with a physiological ligand. Ligands of interest may be drugs or drug candidates or naturally occurring binding partners, physiological substrates etc. Thus, the ligand can bind to the target to form a larger complex. The ligand can bind to the target with any affinity i.e. with high or low affinity. Generally, a ligand which binds to the target with high affinity may result in a more thermally stable target compared to a ligand which binds to the target with a lower affinity. Typically, a ligand capable of binding to a target may result in the thermal stabilization of that target protein by at least 0.25 or 0.5° C. and, for example, at least 1, 1.5 or 2° C. The term “natural” in the context of ligands, as used herein, means any ligand that exists in or is derived from plants, animals, and/or other microorganisms as opposed to compounds or forms of matter that are artificial, synthetic and/or made by chemical synthesis or proteins that are not expressed by their natural origin organism. Derived natural ligands, especially if the ligand is a polypeptide or protein, in the meaning of the present disclosure, demonstrate at least 70%, at least 75%, at least 80%, at least 85%, at least 90%, at least 95%, at least 97% or at least 99% sequence homology with the wildtype ligand over its whole length.

The terms “linked” or “conjugated” as used herein are used interchangeably and are intended to include any or all of the mechanisms known in the art for coupling the reporter molecule to the first or second binding molecule. For example, any chemical or enzymatic linkage known to those with skill in the art is contemplated including those which result from photoactivation and the like. Homofunctional and heterobifunctional cross-linkers are all suitable. Reactive groups which can be cross-linked with a cross-linker include primary amines, sulfhydryls, carbonyls, carbohydrates and carboxylic acids.

The term “reporter molecule” as used herein refers to molecules useful for detecting the presence, intensity or quantity of other molecules that are attached to it, e.g. as a conjugate. These molecules are often called detectable molecules and such phrases are used interchangeably herein. Molecules detectable by spectroscopic, photochemical, biochemical, enzymatic, immunochemical, electrical, radiographic and optical means are known. Optically detectable molecules include fluorescent labels, such as commercially available fluorescein and Texas Red. Detectable molecules useful in the present disclosure include any biologically compatible molecule which may be conjugated to a binding molecule, such as an antibody, without compromising the ability of the binding molecule to interact with the analyte molecule, and without compromising the ability of the reporter molecule to be detected. These include molecules which interact with other molecules as a means of creating a reportable event for example as some reporter molecules used in the known BRET and FRET assays which include fragmented molecular systems. Conjugated molecules (or conjugates) of the binding molecule and detectable molecules are thus useful in the present disclosure. Preferred for attachment to the binding molecule are detectable molecules capable of being easily synthesized, easily conjugated to the binding molecule and easily detected, for example by using a cell phone camera.

In some aspects, the reporter is selected from the group consisting of a dye, a radionuclide, an enzyme and combinations thereof.

The dye can be either a “small molecule” dye/fluors, or a proteinaceous dye/fluors (e.g. green fluorescent proteins and all variants thereof). Suitable dyes include, but are not limited to, 1,1′-diethyl-2,T-cyanine iodide, 1,2-diphenylacetylene, 1,4-diphenylbutadiene, 1,6-Diphenylhexatriene, 2-Methylbenzoxazole, 2,5-Diphenyloxazole (PPO), 4-(dicyanomethylene)-2-methyl-6-(p-dimethylaminostyryl)-4H-pyran (DCM), 4-Dimethylamino-4′-nitrostilbene, 4′,6-Diamidino-2-phenylindole (DAPI), 5-ROX, 7-AAD, 7-Benzylamino-4-nitrobenz-2-oxa-1,3-diazole, 7-Methoxycoumarin-4-acetic acid, 9,10-Bis(phenylethynyl)anthracene, 9,10-Diphenylanthracene, Acridine Orange, Acridine yellow, Adenine, Allophycocyanin (APC), AMCA, AmCyan, Anthracene, Anthraquinone, APC, Auramine O, Azobenzene, Benzene, Benzoquinone, Beta-carotene, Bilirubin, Biphenyl, BO-PRO-1, BOBO-1, BODIPY FL, Calcium Green-1, Cascade Blue™, Cascade Yellow™, Chlorophyll a, Chlorophyll b, Chromomycin, Coumarin, Coumarin 1, Coumarin 30, Coumarin 314, Coumarin 343, Coumarin 6, Cresyl violet perchlorate, Cryptocyanine, Crystal violet, Cy2, Cy3, Cy3.5, Cy5, Cy5.5, Cy7, Cytosine, DA, Dansyl glycine, DAPI, DiI, DiO, DiOCn, Diprotonated-tetraphenylporphyrin, DsRed, EDANS, Eosin, Erythrosin, Ethidium Monoazide, Ethyl p-dimethylaminobenzoate, FAM, Ferrocene, FI, Fluo-3, Fluo-4, Fluorescein, Fluorescein isothiocyanate (FITC), Fura-2, Guanine, HcRed, Hematin, Histidine, Hoechst, Hoechst 33258, Hoechst 33342, IAEDANS, Indo-1, Indocarbocyanine (C3)dye, Indodicarbocyanine (C5)dye, Indotricarbocyanine (C7)dye, LC Red 640, LC Red 705, Lucifer yellow, LysoSensor Yellow/Blue, Magnesium octaethylporphyrin, Magnesium octaethylporphyrin (MgOEP), Magnesium phthalocyanine (MgPc), Magnesium tetramesitylporphyrin (MgTMP), Magnesium tetraphenylporphyrin (MgTPP), Malachite green, Marina Blue®, Merocyanine 540, Methyl-coumarin, MitoTracker Red, N,N′-Difluoroboryl-1,9-dimethyl-5-(4-iodophenyl)-dipyrrin, N,N′-Difluoroboryl-1,9-dimethyl-5-[(4-(2-trimethylsilylethynyl), N,N′-Difluoroboryl-1,9-dimethyl-5-phenydipyrrin, Naphthalene, Nile Blue, Nile Red, Octaethylporphyrin, Oregon green, Oxacarbocyanine (C3)dye, Oxadicarbocyanine (C5)dye, Oxatricarbocyanine (C7)dye, Oxazine 1, Oxazine 170, p-Quaterphenyl, p-Terphenyl, Pacific Blue®, Peridinin chlorophyll protein complex (PerCP), Perylene, Phenol, Phenylalanine, Phthalocyanine (Pc), Pinacyanol iodide, Piroxicam, POPOP, Porphin, Proflavin, Propidium iodide, Pyrene, Pyronin Y, Pyrrole, Quinine sulfate, R-Phycoerythrin (PE), Rhodamine, Rhodamine 123, Rhodamine 6G, Riboflavin, Rose bengal, SNARF®, Squarylium dye III, Stains-all, Stilbene, Sulforhodamine 101, SYTOX Blue, TAMRA, Tetra-t-butylazaporphine, Tetra-t-butylnaphthalocyanine, Tetrakis(2,6-dichlorophenyl)porphyrin, Tetrakis(o-aminophenyl)porphyrin, Tetramesitylporphyrin (TMP), tetramethylrhodamine, Tetraphenylporphyrin (TPP), Texas Red® (TR), Thiacarbocyanine (C3)dye, Thiadicarbocyanine (C5)dye, Thiatricarbocyanine (C7)dye, Thiazole Orange, Thymine, TO-PRO®-3, Toluene, TOTO-3, TR, Tris(2,2′-bipyridyl)ruthenium(II), TRITC, TRP, Tryptophan, Tyrosine, Uracil, Vitamin B12, YO-PRO-1, YOYO-1, Zinc octaethylporphyrin (ZnOEP), Zinc phthalocyanine (ZnPc), Zinc tetramesitylporphyrin (ZnTMP), Zinc tetramesitylporphyrin radical cation, and Zinc tetraphenylporphyrin (ZnTPP). Suitable optical dyes are well-known in the art and described in the 1996 Molecular Probes Handbook by Richard P. Haugland.

In some aspects, the dye may be an Alexa Fluor® dye, including Alexa Fluor® 350, Alexa Fluor® 405, Alexa Fluor® 430, Alexa Fluor® 488, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 647, Alexa Fluor® 660, Alexa Fluor® 680, Alexa Fluor® 700, and Alexa Fluor® 750 (Life Technologies Corporation, 5791 Van Allen Way, Carlsbad, Calif. 92008).

In some aspects, the dye may be a tandem fluorophore conjugate, including Cy5-PE, Cy5.5-PE, Cy7-PE, Cy5.5-APC, Cy7-APC, Cy5.5-PerCP, Alexa Fluor® 610-PE, Alexa Fluor® 700-APC, and Texas Red-PE. Tandem conjugates are less stable than monomeric fluorophores, so comparing a detection reagent labeled with a tandem conjugate to reference solutions may yield MESF calibration constants with less precision than if a monomeric fluorophore had been used.

In some aspects, the dye may be a fluorescent protein such as green fluorescent protein (GFP; Chalfie, et al., Science 263(5148):802-805 (Feb. 11, 1994); and EGFP; Clontech-Genbank Accession Number U55762), blue fluorescent protein (BFP; 1. Quantum Biotechnologies, Inc. 1801 de Maisonneuve Blvd. West, 8th Floor, Montreal (Quebec) Canada H3H 1J9; 2. Stauber, R. H. Biotechniques 24(3):462-471 (1998); 3. Heim, R. and Tsien, R. Y. Curr. Biol. 6:178-182 (1996)), cyan fluorescent protein (CFP), and enhanced yellow fluorescent protein (EYFP; 1. Clontech Laboratories, Inc., 1020 East Meadow Circle, Palo Alto, Calif. 94303). In some aspects, the dye is dTomato, FlAsH, mBanana, mCherry, mHoneydew, mOrange, mPlum, mStrawberry, mTangerine, ReAsH, Sapphire, mKO, mCitrine, Cerulean, Ypet, tdTomato, Emerald, or T-Sapphire (Shaner et al., Nature Methods, 2(12):905-9. (2005)).

In some aspects, the dye may be a fluorescent semiconductor nanocrystal particle, or quantum dot, including Qdot® 525 nanocrystals, Qdot® 565 nanocrystals, Qdot® 585 nanocrystals, Qdot® 605 nanocrystals, Qdot® 655 nanocrystals, Qdot® 705 nanocrystals, Qdot® 800 nanocrystals (Life Technologies Corporation, 5791 Van Allen Way, Carlsbad, Calif. 92008). In some aspects, the dye may be an upconversion nanocrystal, as described in Wang et al., Chem. Soc. Rev., 38:976-989 (2009).

In some aspects, the dye may be an ATTO 390 dye, ATTO 425 dye, ATTO 465 dye, ATTO 488 dye, ATTO 495 dye, ATTO 520 dye, ATTO 532 dye, ATTO 550 dye, ATTO 565 dye, ATTO 590 dye, ATTO 594 dye, ATTO 610 dye, ATTO 611X dye, ATTO 620 dye, ATTO 633 dye, ATTO 635 dye, ATTO 637 dye, ATTO 647 dye, ATTO 647N dye, ATTO 655 dye, ATTO 665 dye, ATTO 680 dye, ATTO 700 dye, ATTO 725 dye and ATTO 740 dye manufactured by ATTO-TEC GmbH (Siegen, Germany).

The term “radionuclide”, as used herein, relates to medically useful radionuclides, including, for example, positively charged ions of radiometals such as Y, In, Cu, Lu, Tc, Re, Co, Fe and the like, such as ⁹⁰Y, ¹¹¹In, ⁶⁷Cu, ⁷⁷Lu, ⁹⁹Tc and the like, e.g., trivalent cations, such as ⁹⁰Y and ¹¹¹In.

Examples of reporter enzymes which can be used to practice the disclosure include hydrolases, lyases, oxidoreductases, transferases, isomerases and ligases. Some examples are phosphatases, esterases, glycosidases and peroxidases. Specific examples include alkaline phosphatase, lipases, beta-galactosidase and horseradish peroxidase. In other aspects of the disclosure, the reporter is horseradish peroxidase.

In various aspects of the disclosure, the reporter molecule is selected from the group consisting of protein, e.g., an enzyme, e.g., horseradish peroxidase, nucleotide, dye, gold, silver and platinum.

The terms “at least one” and “plurality”, as interchangeably used herein, relate to one or more, in particular 1, 2, 3, 4, 5, 6, 7, 8, 9, 10, 50, 100, 500, 1000 or more.

The term “attached”, as used herein, refers to the binding of one element to another one. The term is to be understood in a broad sense including covalent and non-covalent binding of the two elements. In some aspects, the binding is non-covalently.

In some aspects of the disclosure, the layer composition of the unit of stacked layers is as follows: sample layer; at least one conjugation layer; at least one blocking layer; absorption layer, and the flow direction of the sample is from the sample layer to the absorption layer. The term “flow direction”, as used herein, relates to flow of the liquid including the analyte molecule through the unit of stacked layers. The flow direction is said to be “from X to Y”, when the liquid including the analyte molecule first contacts X and subsequently contacts Y.

In some aspects, each of the sample layer, the at least one conjugation layer, the at least one blocking layer and the absorption layer are separated by a separation layer comprising a membrane. Such separation is depicted in FIG. 1. The separation layer may consist of or comprise any material that does not interfere with the flow of the liquid containing the analyte molecule. Interference in the context may be the blocking of the flow of the liquid or an unspecific interaction with any component of the liquid, such as the analyte molecule. In various aspects, the separation layer is made of cotton.

Also included within the scope of the present disclosure is that the unit of stacked layers further comprises a stop layer that is, in flow direction, immediately located behind the blocking layer, wherein the stop layer is dissolved upon contact with the sample. The stop layer may consist of or comprise a salt or polymer. Usually, the stop layer has such density that it cannot be passed or at least provide a barrier for the analyte molecule. The term “dissolved”, as used herein, means that the stop layer dissolves in sufficient quantity to cause a significant flow of the analyte molecule to the next layer. Such resolution of the stop layer may be determined at 25° C. and under normal atmosphere pressure. In some aspects, 50% of the stop layer are dissolved after at least 10 seconds, at least 20 seconds, at least 30 seconds, at least 1 minute, at least 1.5 minutes, at least 2 minutes, at least 3 minutes, at least 5 minutes or at least 10 minutes. As used herein, “salt” is an ionic compound in which the proportions of the ions are such that the electric charges cancel out, so that the bulk compound is electrically neutral. “Inorganic salt” includes salts include, for example, oxides, carbonates, sulfates, and halides. The halides include fluoride (F), chloride (Cl⁻), bromide (Br⁻), iodide (I⁻) and astatide (At⁻). Inorganic halide salts include, for example, sodium chloride (NaCl), potassium chloride (KCl), potassium iodide (KI), lithium chloride (LiCl), copper(II) chloride (CuCl₂), silver chloride (AgCl), and chlorine fluoride (ClF).

A “polymer” as used herein refers to a macromolecular organic compound that is largely, but not necessarily exclusively, formed of repeating units covalently bonded in a chain, which may be linear or branched. A “repeating unit” is a structural moiety of the macromolecule which is found more than once within the macromolecular structure. Typically, a polymer is composed of a large number of only a few types of repeating units that are joined together by covalent chemical bonds to form a linear backbone, from which substituents may or may not depend in a branching manner. The repeating units can be identical to each other but are not necessarily so. Therefore a structure of the type -A-A-A-A- wherein A is a repeating unit is a polymer, also known as a homopolymer, and a structure of the type -A-B-A-B- or -A-A-A-B-A-A-A-B- wherein A and B are repeating units, is also a polymer, and is sometimes termed a copolymer. A structure of the type -A-A-A-C-A-A-A or A-B-A-C-A-B-A wherein A and B are repeating units but C is not a repeating unit (i.e., C is only found once within the macromolecular structure) is also a polymer under the definition herein. When C is flanked on both sides by repeating units, C is referred to as a “core” or a “core unit.” A short polymer, formed of up to about 10 repeating units, is referred to as an “oligomer.” There is theoretically no upper limit to the number of repeating units in a polymer, but practically speaking the upper limit for the number of repeating units in a single polymer molecule may be approximately one million. Polymers that are dissolved upon the contact with different liquids, such as water-based or ethanol-based liquids or combinations thereof, are well known in the art.

In some aspects of the disclosure, the membrane of the at least one conjugation layer and the first binding molecule are non-covalently attached to each other. “Non-covalent” as used herein refers to one or more electrostatic, hydrophilic, or hydrophobic interactions. Such non-covalent interaction of the conjugation layer and the binding molecule may be achieved by incubating the conjugation layer and a solution comprising the binding molecule together and by drying said solution. However, the skilled person is well-aware of alternative methods to attach the first binding molecule non-colavently to the conjugation layer.

In some aspects, the plurality of analyte molecules is covalently immobilized on the membrane of the at least one blocking layer. “Covalent binding” as used herein refers to two moieties (for instance the analyte molecule and the blocking layer) that are attached by at least one bond. Covalent bonds may be formed directly between said elements or may be formed by a cross linker or by inclusion of a specific reactive group on either of said elements or both. Immobilization may include a combination of covalent and non-covalent interactions.

In some aspects, the absorption layer further comprises a substrate for the reporter molecule (or simply a reporter). Reporter substrate, as used herein, is intended to include any substrate capable of being acted on by the reporter. For example, the interaction between the reporter and the reporter substrate produces a qualitative or quantitative effect. A “reporter substrate” as used herein is a substrate (or substrates) that is particularly adapted to facilitate measurement of either the disappearance of a substrate or the appearance of a product in connection with a catalyzed reaction. Reporter substrates can be free in solution or bound (or “tethered”), for example, to a surface, or to another molecule. A reporter substrate can be labelled by any of a large variety of means including, for example, fluorophores (with or without one or more additional components, such as quenchers), radioactive labels, biotin (e.g. biotinylation) or chemiluminescent labels. In case the reporter is horseradish peroxidase, the substrate is, for example, luminol.

In some aspects of the disclosure, the unit of stacked layers comprises at least 6, at least 7, at least 8, at least 9 or at least 10 blocking layers.

In some aspects, the analyte molecule, virus or cell of interest is selected from the group consisting of cell, protein, virus, viral toxin, bacterial toxin, biotoxin, parasite, fungus, nucleotide and natural ligand.

The term “cell”, as used herein, refers to the basic structural, functional, and biological unit of all known living organisms. A cell is the smallest unit of life that can replicate independently. Cells comprise cytoplasm enclosed within a membrane, which contains many biomolecules such as proteins and nucleic acids. Organisms can be classified as unicellular (consisting of a single cell; including bacteria and viruses) or multicellular (including plants and animals).

In a second aspect, the present disclosure is directed to a method to prepare the device of the disclosure, comprising: incubating and drying the membrane of the at least one conjugation layer and a sample comprising the first binding molecule; immobilizing, e.g., covalently immobilizing, the plurality of analyte molecules, virus or cells of interest on the membrane of the at least one blocking layer; and stacking the layers of the unit of stacked layers to form the device of the disclosure.

In a further aspect, the disclosure relates to the use of the device of the disclosure to determine or quantify the presence of an analyte molecule, virus or cell of interest in a sample.

In some aspects, chemiluminescence, fluorescence, colorimetry, or electrochemistry in combination with a dedicated reader or a cell phone app are used for the determination or quantitation of the presence of the analyte molecule, virus or cell of interest.

Further, the present disclosure is directed to a method to determine or quantify the presence of an analyte molecule, virus or cell of interest in a sample, comprising: contacting the sample and the device of the disclosure; and determining or quantifying the presence of the analyte molecule by detecting a reporter molecule dependent signal.

The term “contacting”, as used herein, refers generally to providing access of one component, reagent, analyte or sample to another. For example, contacting can involve mixing the device of the disclosure with a sample comprising the analyte molecule. This reaction may comprise one or more components, reagents, analytes or samples, such as dimethyl sulfoxide (DMSO) or a detergent, which facilitates mixing, interaction, uptake, or other physical or chemical phenomenon advantageous to the contact between cell/sample and bacterial derivative/composition.

Finally, the present disclosure relates in a fifth aspect to a kit of parts comprising at least one device of the disclosure.

In some aspects, the kit comprises: a first device of the disclosure that detects or quantifies an analyte molecule or cell of interest that is indicative for pregnancy; and a second device of the disclosure that detects or quantifies an analyte molecule, virus or cell of interest that is indicative for Zika-virus (ZIKV) infection.

“Pregnancy”, also known as gravidity or gestation, is the time during which one or more offspring develop inside a female. The term “Zika-virus”, as used herein, refers to a member of the virus family Flaviviridae. It is spread by daytime-active Aedes mosquitoes, such as A. aegypti and A. albopictus. The infection, known as Zika fever or Zika virus disease, often causes no or only mild symptoms, similar to a very mild form of dengue fever. Zika can also spread from a pregnant woman to her fetus. This can result in microcephaly, severe brain malformations, and other birth defects. Zika infections in adults may result rarely in Guillain-Barré syndrome.

In some aspects of a kit of parts, the analyte molecule that is indicative for pregnancy is human chorionic gonadotropin (hCG) or fragments thereof.

The term “human chorionic gonadotropin (hCG)”, as used herein, refers to a hormone produced by the placenta after implantation. The presence of hCG can be detected in some pregnancy tests (HCG pregnancy strip tests). Some cancerous tumors produce this hormone; therefore, elevated levels measured when the patient is not pregnant can lead to a cancer diagnosis and, if high enough, paraneoplastic syndromes. However, it is not known whether this production is a contributing cause or an effect of carcinogenesis.

In some aspects of a kit of parts, the analyte molecule that is indicative for Zika-virus (ZIKV) infection is a Zika-virus virion, a Zika-virus protein, e.g., the NS1 protein, a Zika-virus nucleotide and/or fragments thereof.

The term “virion”, as used herein, refers to a single, stable infective viral particle that is released from the cell and is fully capable of infecting other cells of the same type.

In some aspects of the kit, the at least one device of the disclosure detects or quantifies an analyte molecule, or cell of interest that is indicative for (a) influenza A/B infection, respiratory syncytial virus (RSV) infection, parainfluenza infection, adenovirus infection and/or metapneumovirus infection; (b) dengue virus infection, Zika virus infection, malaria infection, lassa virus infection, ebola virus infection, west-nile virus infection and/or yellow fever virus infection; (c) fertility; or (d) diarrhea, travel fever, child fever, meningitis/encephalitis, respiratory diseases, sepsis, hemorhhagi fever and/or cancer.

The term “fragments therefore”, as used herein, relates to fragments of a polypeptide, protein or nucleotide therein said fragment has at least 70%, at least 75%, at least 80%, at least 85%, at least 90%, at least 95%, at least 97% or at least 99% sequence homology with the non-fragmented wildtype molecule over its whole length.

The devices of the disclosure used in the kit may enabling multiplex detection of target analytes. Several individual units of stacked layers (StackPads) may be encased in a plastic (or other material) enclosure. The individual stacks are connected to a common container, in which the sample can be filled. Once the user puts the sample in the container, the sample is distributed among the individual stacks via capillary flow. In alternative aspects, each unit of stacked layers can have its own container. The purpose of the multiplex system is to combine several stacks that test for different markers (i.e., different pathogen proteins, different blood markers, different pathogens or combinations) in one device that has a small footprint and allows for a rapid and hassle free handling. Compared to existing technologies (mainly lateral flow) the present technology can be arranged as a multiplexing system. The mere size of prior art technology and the required sample volume make it impractical and not useful to arrange it as a multiplexing system. Because of the small size and small needed volume of the present technology, several stacks can be grouped in one container, and even more than 10 devices can be used at once making the present system efficient and practical.

Thus, in some aspects of the disclosure, the device comprises at least three units of stacked layers.

In one aspect, the stacks will be shaped like a cylinder, and multiple cylinders with different radius can be assembled into one unit allowing for testing multiple analytes at the same time or as internal control or positive control. Sides of the cylinders will be coated with a hydrophobic material (for example wax), that will prevent leaching into neighboring cylinders.

In one aspect, the method to detect an analyte molecule, virus or cell can include electrochemical detection. In this aspect, in the top layer above the absorption pad an electrochemical electrode will be included comprising a working electrode, reference and/or counter electrode. In this aspect, a substrate enzyme combination may be used that will enable an electrochemical reaction that can be measured with a potentiostat reader device.

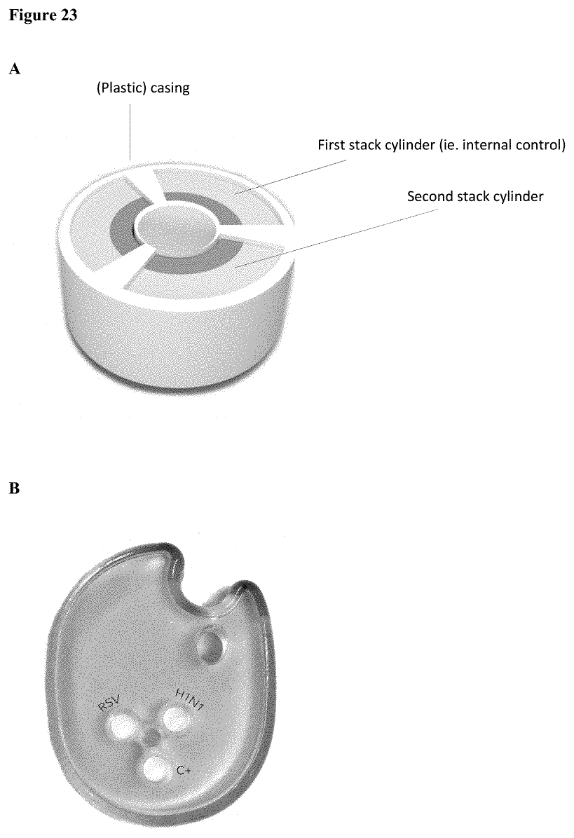

In order to prevent uncontrolled flow of the (sample) liquid in the casing/membrane interface several techniques may be employed: 1) the outer membrane surface may be coated with a coating that will prevent uncontrolled capillary flow, such as wax, silicone or other material; 2) the paper stacks may have no physical contact with the (plastic) casing and may be positioned so that there is no uncontrolled capillary flow between the membrane end and (plastic) backing. In order to secure two alternative approaches are presented: 1) a needle positioned on top of the stack and only contacting the top layer may ensure the right pressure to keep the stacks in shape and prevent movement as well as prevent formation of bubbles between the stacks (cf. FIG. 23 (A)). In the second alternative, a needle puncturing all layers of the unit of stacked layers may prevent disassembly of the layers and enables right positioning.

In one aspect, the stacks may be used in conventional 24, 48, 96 or 384-well plates for high-throughput screening and testing. Each Stack pad will be put in one well. The results may be read with a conventional ELISA plate reader or luminometer, depending on the detection method of choice (chromogenic or luminescence).

### Examples

### Materials and Methods

### Reagents

Phosphate-buffered saline (PBS) tablets (cat. no. P4417) were purchased from Sigma-Aldrich. PBS-0.05% (v/v) Tween (PBST) was prepared by adding 0.5 mL of Tween-20 solution (cat. no. P7949) to 1 L of PBS buffer. The 5% (w/v) skim milk (SM) solution was prepared by adding 5 g of SM powder (70166) to 100 mL of PBST solution. Milli-Q ultrafiltered (UF) H₂O (with a resistivity of 18.2 M Q cm at 25° C.) was used in the preparation of all solutions.

### Immunoreagents

Rabbit monoclonal anti-E. coli antibody (IgG), with conjugated HRP enzyme (ViroStat, cat. no. VIR-1004) or without (ViroStat, cat. no. VIR-1001), was purchased from ENCO, Israel.

### Bacterial Strain Growth and Maintenance

Five different bacteria strains were used in this study: four E. coli strains (DH5α, K12, B, and MC1061) and Salmonella typhimurium. Prior to measurements, all strains were cultivated in 10 mL of clear LB. Bacteria were grown overnight at 37° C. in a rotary thermoshaker (Gerhardt, Konigswinter, Germany) at 120 rpm. Cultures were then diluted to approximately 107 cells mL⁻¹ and regrown in 25 mL of LB at 26° C., without shaking, to an early exponential phase (0.2 OD₆₀₀ₙₘ) as determined by an Ultrospec 2100 pro spectrophotometer (Amersham, Bucks, U.K.). The cultures were then centrifuged (MiniSpin plus, Eppendorf, Germany) at 12 000 rpm for 5 min. After replacing the supernatant with PBST buffer, bacteria were homogenized by gentle pipetting. This step was repeated thrice. The bacteria were then diluted to the final testing concentrations.

### Device Fabrication

### Membranes

Sample (cat. no. GFBR4), absorbent (cat. no. AP-080), and conjugate release matrix (cat. no. PT-R5) pads were purchased from Advanced Microdevices Pvt. Ltd. (India). Amersham protran nitrocellulose membrane 0.45 μm (cat. no. 10600044) was purchased from GE-Healthcare.

### Assay Reagent Immobilization Procedures

Substrate pads were made by cutting 6 mm diameter pads from absorption pads, wetting with 100 μL of luminol—H₂O₂ substrate solution (ratio 1:1) (cat. no. 1705040, BioRad), and drying for 3 h at 37° C. in the dark. Conjugation pads (D=6 mm) were prepared by wetting pads with 80 μL of anti-E. coli—HRP antibodies (diluted with PBST [PBS (0.203 g L⁻¹ NaH₂PO₄, 1.149 g L⁻¹ Na2HPO₄, 8.5 g L⁻¹ NaCl) (pH 7.2) with 0.05% (v/v) Tween-20] and dried for 2 h at 37° C. The conjugate pads were stored with desiccant gel at room temperature. Blocking layers were made by exposing the nitrocellulose sheets to bacteria (diluted in PBS) for 1 h. The sheets were then washed thrice with PBST and incubated at room temperature with blocking solution [PBS, 5% (w/v) SM and 0.05% (v/v) Tween-20] for 1 h. The nitrocellulose sheets were then washed thrice before drying at 30° C.

### Assembly of the Stacked Immunoassay System

The stacked immunoassay was assembled by placing all prepared pads one on top of another in the following order (FIG. 1). The sample, conjugated, blocking, and absorbent (substrate) pads were stacked from bottom to top in this order. Each layer was separated with a sample pad to encourage a directed flow toward the center of the pads and provide space for the conjugation. Finally, in order to provide a flow process with directional integrity, all stacked pads were placed in plastic holders.

### Measurement Procedure

Each plastic holder (with all pads in the right order) was placed above a 150 μL water sample. Water was observed to seep through the sample pads and migrate, by capillary force, from the lowest to the uppermost layer. Target analytes first diffuse within the layer with the labeled antibacterial antibodies, then move on through the blocking layer to the absorption pad containing the dried substrate for use by the marker enzyme.

### Instrumentation

In samples containing the target bacteria, the light signal produced was captured with a CCD camera (Retiga-SRV FAST 1394, InterFocus, U.K.). The CCD camera was placed 30 cm above the stacks, and serial pictures of 15 s of exposure time were taken with QCapture pro software. Measurement occurred 5 min after the stack configuration was exposed to the liquid sample.

### Optimization Steps

### Immobilization Procedure

The effect of the analyte immobilization procedures in enabling the capture of the arriving nonconjugated labeled antibodies on the blocking membrane was evaluated by exposing 1 cm×2 cm nitrocellulose sheets to anti-E. coli—HRP antibodies (1:2000 dilution) solution diluted in PBST. Three different immobilization approaches were tested: incubation, drop deposition, and spreader. In the case of the incubation procedure, nitrocellulose sheets were exposed to 2 mL of antibody solutions for different time periods (15, 30, and 60 min), and thereafter rinsed thrice with PBST before incubating with a blocking solution for 1 h. The sheets were again rinsed thrice with PBST and dried at room temperature. In the drop approach, 1 mL of each antibody solution was carefully deposited at the center of a 1 cm×2 cm nitrocellulose sheet and dried at room temperature. For the spray approach, a chromatography dispenser (Z529710, Sigma-Aldrich) was used to dispense 0.1 mL s⁻¹ antibodies on the 1 cm×2 cm membrane. Three spread durations were tested (10, 20, and 30 s).

### Leaching Issues

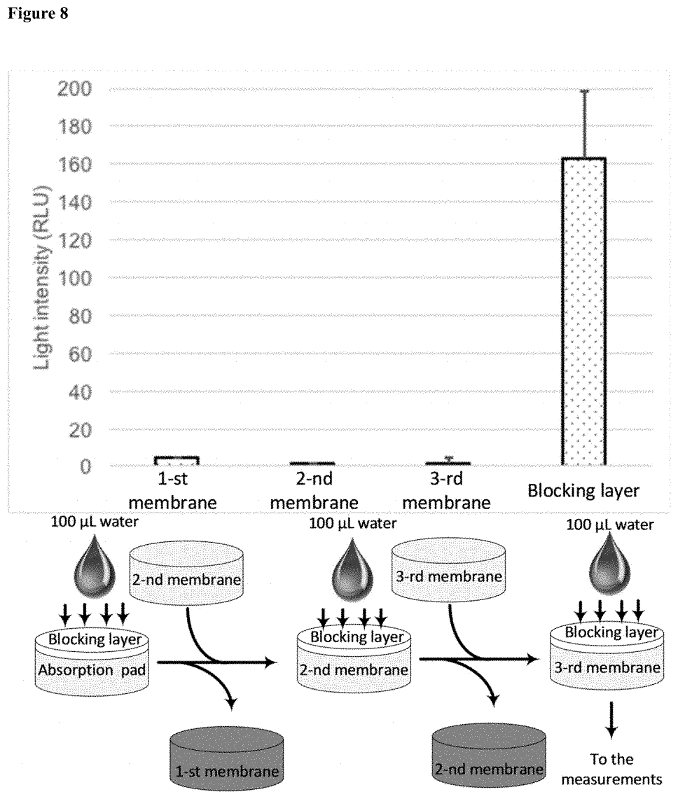

To prevent false positive responses, immobilized bacteria must be strongly attached onto the nitrocellulose membrane and should capture any unbound antibodies during the assay operation. DH5α bacteria (106 cells were immobilized on the membrane (Immobilization Procedure section) to create such a blocking layer. Its efficiency was evaluated with the following experiment. Modified membranes (with the bacterial target strain immobilized) were cut into circles of 6 mm in diameter and placed on absorption pads (FIG. 8). An amount of 100 μL of an antibody solution (1:4000 dilution in PBST) was flowed through the blocking layer to the absorption pad. The absorption pad was replaced with a fresh/dry one, and another 100 μL of antibodies solution was thus transferred. This step was repeated thrice, and all pads (three adsorption and one blocking) were placed into separate wells of a white opaque 96-well microtiter plate. A 100 μL substrate solution [luminol—H₂O₂ (ratio 1:1)] was added to each well, and the light activity was measured with a Luminoskan Ascent luminometer (Thermo Fisher Scientific, U.S.A.).

### Specificity Testing

Two different approaches (ELISA and direct measurements on nitrocellulose pads) were used to determine the specificity of the commercial antibodies to the model E. coli DH5α strain of interest. Four different bacterial strains were likewise immobilized on nitrocellulose membranes as previously described in the Immobilization Procedure section. Thereafter, each membrane was incubated with anti-E. coli—HRP solution (1:2000) for 1 h. The membranes were then rinsed thrice with PBST and dried at room temperature. Prior to measurement, 100 μL of substrate solution [luminol—H₂O₂ (ratio 1:1)] was pipetted onto each membrane and light activity recorded with a CCD camera.

ELISA measurements were done using 100 μL of the anti-E. coli antibodies solution, which were added into each well of a 96-well microtiter plate (MaxiSorp, Nunc). The plate was sealed to avoid evaporation, and the antibodies were allowed to adsorb overnight at 4° C. After incubation, the coating buffer was decanted and the plate was washed with PBS. An amount of 150 μL/well of PBST-SM blocking solution at pH 7.2 was added to reduce the overall background and increase the sensitivity of the assay. The plate was then incubated for 1 h at 37° C. and the wells were washed thrice with PBST-SM. Amounts of 100 μL of different bacterial strains (10⁶ cells mL⁻¹) were added to each well in triplicates before incubation for 1 h at 37° C. Additional empty wells and noncoated wells were used to check the level of background signal. The wells were washed thrice with PBST-SM (pH 7.2), 100 μL/well of a solution of anti-E. coli peroxidase-labeled antibodies (1:2000) was added, and the plate incubated for 1 h at 37° C. After incubation, the wells were again washed thrice with PBST-SM (pH 7.2), then before each measurement with a Luminoskan Ascent luminometer, 150 μL of oxidizing reagent (H₂O₂) and enhancer luminol reagent solutions were injected. The data were collected, and the mean and standard deviation of triplicates were calculated for each point with signals reported in RLU (relative light units).

### Determining the Volume of Sample

To determine the final sample volume that will be used in future experiments, StackPad setups were exposed to different volumes (50, 60, 70, 80, 90, 100, 110 and 120 μL) of colored thionine solutions and photographed for 5 min after the addition of liquid.

### Optimization Blocking Efficiency of the Immunoassay

The blocking efficiency of the stack pad biosensor was optimized in testing three configurations (zero, three, and six blocking layers), modified with various antibody concentrations (1:1000, 1:2000, and 1:5000 dilution), all immobilized as described in the Immobilization Procedure section. A cotton membrane is placed to separate each blocking layer so as to prevent nondirected flow. In a control setup, nitrocellulose membranes were not modified with immobilized bacteria. Prior to setup assembly, these membranes were blocked with the same PBST-SM blocking solution [5% (w/v) SM in PBST] followed by complete drying. Each stack pad configuration was exposed to 150 μL of either pure water or spiked with DH5α bacteria (10⁶ cell mL⁻¹). The signal generated was captured with a CCD camera, 5 min after sample addition, as previously mentioned (Instrumentation section).

### First Whole-Setup Experiments

After optimization, the final setup conformation was established. The order of the pads in the plastic holder was (from lower to upper layers) sample pad, conjugation pad (with anti-E. coli antibodies conjugated to HRP), six blocking layers (with 10⁶ cells mL⁻¹ DH5α cells), and absorption pad (with dry substrate). To prevent nondirected liquid flow out of the layers, each active layer was separated with a pad made from cotton. Two different samples were tested, clear water and spiked with DH5α bacteria (10⁶ cell mL⁻¹). The stack pads were placed above a 150 μL sample solution and measured with CCD camera 5 min after sample addition.

### Sensitivity of the Stack Pad Immunoassay

The assay sensitivity to the DH5α cells target bacteria was compared with that obtained from ELISA test results. Both approaches were exposed to different DH5α cell concentrations (10¹, 10², 10³, 10⁴, 10⁵, 10⁶ cells mL⁻¹ and pure water). All immobilization procedures and pad preparation were previously described in the Immobilization Procedure section. The setup design was explained in the Optimization Blocking Efficiency of the Immunoassay section, where six blocking layers and a 1:2000 anti-E. coli—HRP dilution were used. The stack pads were placed above a 150 μL bacterial sample solution, and measurements taken with a CCD camera 5 min after sample addition.

### Specificity of the Stack Pads Biosensor

Five different bacterial strains, four E. coli (DH5α, K12, B, and NC1061) and one of S. typhimurium were used. Stack pad design and membrane preparation steps were the same as in the previous section. Each strain was diluted with PBST to 10⁶ cells mL⁻¹ prior to exposure to the stack pad sensor. Amounts of 150 μL of the different bacterial solutions were added to the stack pad, and then measurements taken with a CCD camera 5 min after sample addition.

### Reproducibility of the System

For each development step, a minimum of 20 separate and different stacks were exposed to the tested parameters. Reproducibility in the results can be estimated by the standard deviation values in the figures.

### Detection Using an Optimized StackPad Setup

After optimization, the final multiple membrane setup conformation was established. The order of the pads in the plastic holder is (from lower to upper layers), sample pad, conjugation pad (with anti-E. coli antibodies conjugated to HRP), six blocking layers (with 10⁶ cells mL⁻¹ DH5∝∝ cells) and absorption pad (with dry substrate). To prevent undirected liquid flow out of the layers, each active layer was separated with a pad made from cotton. Two different samples were tested, clear water and one spiked with DH5∝ bacteria (10⁶ cell mL⁻¹). The stackpads were placed above a 150 μL sample solution and measured with a cell-phone camera 5 min after sample addition.

### ELISA as a Bench Mark for the Sensitivity of the StackPad

The immune assay sensitivity to the target DH5∝ cells bacteria was compared with that obtained with ELISA test. Both approaches were exposed to different DH5∝ cell concentrations (10¹, 10², 10³, 10⁴, 10⁵, 10⁶ cells mL⁻¹ and pure water as negative control). All immobilization procedures and pad preparation are described in the aforementioned sections of the methodology. ELISA detection (after optimization) was done as follows: 100 μL of anti-E. coli antibody solution was added into each well of a 96-well microtiter plate (MaxiSorp, Nunc), sealed to avoid evaporation, and antibodies were allowed to coat the wells overnight at 4° C. After incubation, the coating buffer was decanted and the plate rinsed with PBS.150 μL/well of PBST-S.M blocking solution at pH 7.2 was added to reduce the overall background and increase the sensitivity of the assay. The plate was then incubated for 1 h at 37° C. and the wells washed thrice with PBST-S.M. 100 μL of different bacterial concentrations (10¹, 10², 10³, 10⁴, 10⁵ and 10⁶ cells mL⁻¹) were added to each well in triplicates before incubation for 1 h at 37° C. Additional empty wells and non-bacteria coated wells allowed to check the level of background signal. The wells were washed thrice with PBST-S.M. (pH7.2), 100 μL/well of anti-E. coli peroxidase-labeled antibodies (1:2,000 dilution rate) was added, the plate incubated for 1 h at 37° C., washed thrice with PBST-SM (pH 7.2) then 100 μL of substrate 3,3′,5,5′-Tetramethylbenzidine was added into each well, the plate incubated at room temperature for 30 min in the dark, at which point 50 μL/well of 2NH₂SO₄ was added to stop the chemical reaction. The absorbance at 450 nm was determined with the Labsystems Multiscan RCELISA reader after 10 s of shaking. The data were collected by using Labsystems Transmit software, the mean and the standard deviation of the triplicates were calculated for each point and the signal reported in OD450 units (optical density at 450 nm).

### Environmental Samples Tested

Experiments with environmental samples were performed using water collected from the Amazone (French Guiana), Sea of Galilee (Israel, Lat. 32° 86′12.40″ N Lon.35° 53′67.59″ E) and Lachish river (Israel, Lat.31° 48′56. 68″ N Lon.34° 38′35.66″ E) with the pad based biosensor system. Collected water has been stored at 4° C. to reduce temporal quality loss. In addition, stackpads were exposed to spiked DH5-α cells (10³ cfu×mL⁻¹) and plain water samples used respectively as positive and negative controls. Validation of the presence of the bacteria in the tested water samples was carried out using the viable plate method. Samples were plated onto LB agar plates and the live cells were observed following an 24 h incubation at 37° C.

### Image Analysis

Pad color intensity, recorded in JPEG files, was analyzed with ImageJ software (US National Institutes of Health). Colored pad images were transformed to a 32-bit format and converted into grey contrast. Thereafter, the average of the pixel intensities was analyzed for each tested pad.

### Example 1: A First General Principle

In the first step, the analyte is collected and applied so that it is allowed to traverse through a first paper pad until it reaches the confined dried and immobilized horseradish peroxidase (HRP)-conjugated antianalyte capture antibody lying in wait to conjugate the analyte after molecular recognition. Thereafter, the conjugate analyte-antibody complex migrates up to the next “blocker” layer (FIG. 1) where a nitrocellulose membrane has been modified with the immobilized analyte of interest, which may be, for example, Escherichia coli bacteria. If the sample tested is contaminated with the bacteria in question, then the complex moves on. If the target analyte is absent, then the HRP-labeled antibodies will simply bind in this new layer where the cells have been pre-immobilized as the blocking layer. This means that these labeled markers will not diffuse further. Analyte-positive complexes will migrate to the highest pad, where signal measurement occurs (by chemiluminescence, colorimetry, or electrochemistry).

### Example 2: Determination of Optimal Operating Conditions of the Stacks Sensor

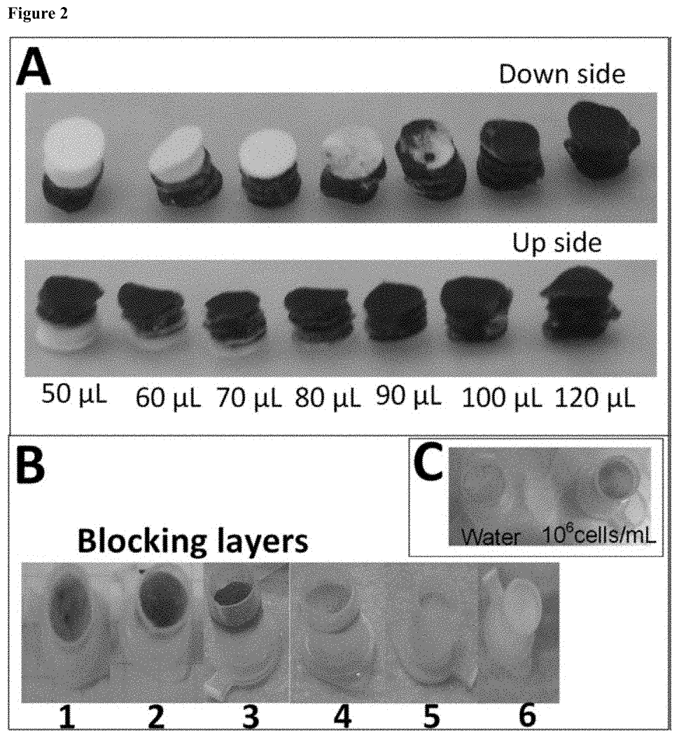

Nitrocellulose membranes were modified by immobilizing the target analyte (in this case E. coli DH5 cells) and then blocking with skim milk. This is an important step that will help prevent a false positive response, therefore, its optimization is critical. It was started by optimizing the sample volume when the stack pad configurations (formation as on FIG. 1, only with six blocking layers separated with cotton pads) were exposed to different volumes of water colored with thionine. Seven different sample volumes were evaluated, and that of 120 μL, was deemed sufficient for the liquid to fully wet the entire membrane stack (FIG. 2A), as seen by the color stains of the different layers inside the stack. With lower volumes (from 50 to 80 μL), the liquid capacity was insufficient to diffuse through the whole setup and only from 90 μL onwards, will liquid reach the upper layers. However, only at 120 μL did the upper (absorption) pad be homogeneously colored, suggesting that 120 μL is the minimum volume that must be used in the operation.

The next optimization step was to determine the optimal number of capture-blocking layers (FIG. 2B) needed to ensure that unbound HRP-specific antibodies to the target entity would be filtered out of the seeping sample liquid as it reaches the uppermost layer. Each setup included the anti-E. coli antibodies conjugated to the horseradish peroxidase (in the conjugation pad), substrate (in adsorption pad) and an incremental number (1-6) of nitrocellulose membranes with immobilized cells thereupon. A working assumption is that the concentration of antibodies must be lower than the available binding epitopes on the immobilized bacteria constituting the blocking layer (FIG. 2B). The more layers present, the greater the capture efficiency, which will be measured by color intensity on the uppermost layer. Indeed, 6 layers were deemed best (FIG. 2B) and adopted in further experiments.

After optimization, the final system setup was exposed to both spiked (DH5-α strain (10⁶ cell/mL)) and uncontaminated water (FIG. 2C), where color intensity in negative control was much lower than that exposed with bacteria. These results confirmed the primary assumption that the blocking layer will filter out the unbound (free) antibodies interacted. The sample with bacteria, have conjugated the labeled antibodies and therefore cannot interact with the next capture layer, and therefore continue to the uppermost layer to produce a color change. However, slight color change was observed with clear water, either because some antibodies managed to avoid the blocking layer or a leaching of the immobilized bacteria (with attached antibodies) occurred from the nitrocellulose membrane.

### Example 3: Effect of Drying Different Volumes of the Reporter

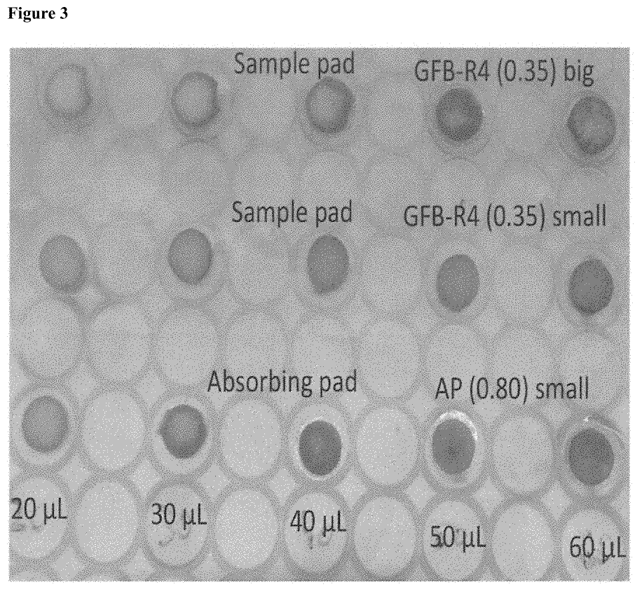

Different volumes of gold nano beads were placed above three different pads types (FIG. 3). Two GFP-R4 nitrocellulose membranes (0.35 mm cutoff, with different diameter) and AP 080 (0.80 mm cutoff, absorbent) were used (FIG. 3). FIG. 3 demonstrates the effect of drying the gold solution on different pads. Lower volumes (20 to 30 μL) create non-uniform rings at the circumference of the pads after drying, which may affect the sample flow and the reaction between target analyte and reporter molecule. Diameter of the pads and its composition also has an effect on the gold composition during the drying process. The pad made from an absorbing pad (0.80 mm) and lower pad diameters showed the best uniformity after drying. It is also important to note that deposition and drying processes were done on hydrophobic surfaces to prevent the leaking of the gold solution from the pads.

### Example 4: Number of Blocking Layers Required to Reduce a False Positive Response by the Assay

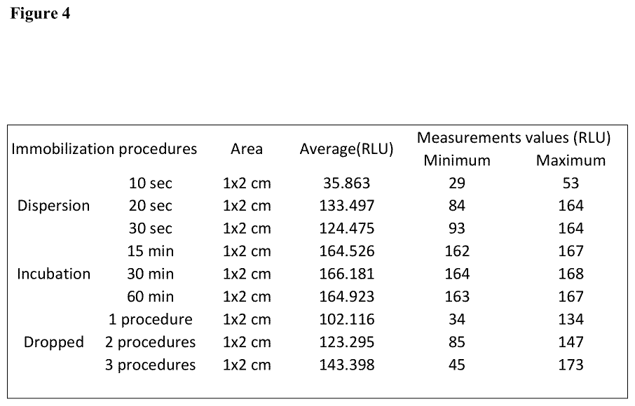

In the proposed system, a hydrophobic nitrocellulose membrane modified with immobilized ‘target’ DH5α E. coli bacteria acts as the capture layer. So, it is crucial that the immobilized capture target bacteria cover the membrane in a homogeneous way, so as to prevent unwanted HRP-antibodies diffusion from occurring though to the next layer. Therefore, it was imperative to closely optimize the blocking capture layer procedure, and their capture efficiency tested after three methods of bacterial blocking depositions. 1× 2 cm membranes strips were incubated after immersion in an antibody solution, or using a spray from chromatography dispenser to deposit the bacteria and finally, adding a bacterial solution drop. Thereafter, the membranes were dried at room temperature, and the coverage tested after the addition of the substrate (luminol and peroxide) triggering the light signal (FIG. 4). Efficiency in the immobilization procedure was checked using three parameters (e.g., minimum light value, maximum light value, and the overall signal average), which are correlated to the final density of the biological capture entities on the nitrocellulose membrane. Despite, small differences between maximum and minimum light values and their proximity to the overall average results, the higher the density of the immobilized antibodies on the capture membrane, the better the immobilization process. From all three methods, only the immersion approach has shown similarity in all parameters, suggesting the creation of a homogeneous blocking layer. Moreover, as similar signals were observed for all incubation periods, it suggests a very fast immobilization process. The worst system, which may produce false positive responses, was the addition of a drop to the membrane as it seems not to diffuse homogeneously during the drying process, even with larger drop volumes.

### Example 5: Effect of the Blocking Pad on the Flow of the Reporter Molecule

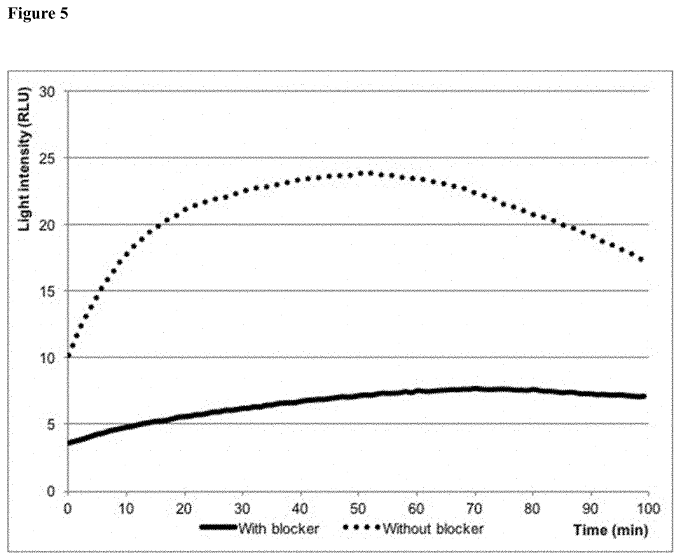

In the next step, the capability of the blocking pad has been demonstrated in stopping the analyte flow to lower sensor layers. This was done by immobilizing the blocking pad with anti-rabbit IgG antibodies and the addition of sample rabbit antibodies with HRP. In the lowest pad, luminol-H₂O₂ (ratio 1:1) was absorbed and dried. FIG. 5 shows the stopping effect of the blocking layer.

### Example 6: Effect of the IMP Concentration

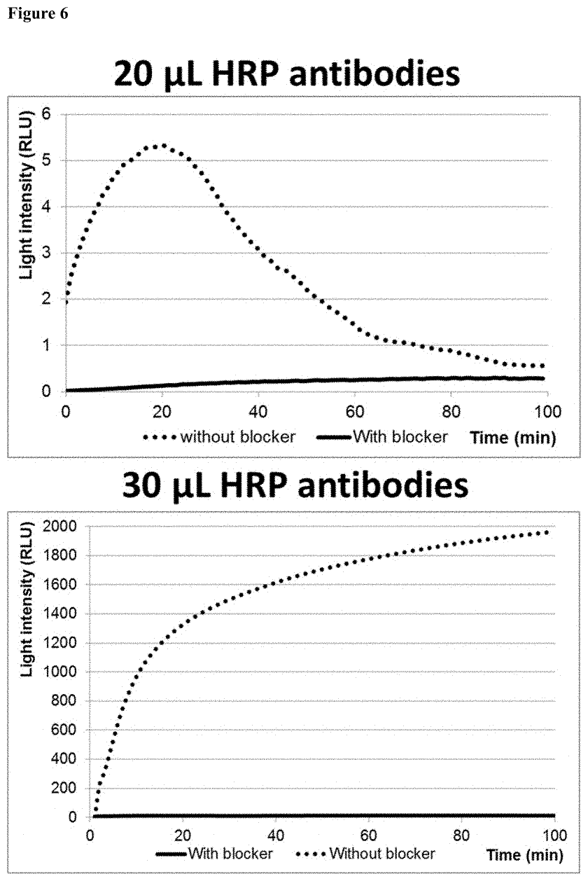

Immobilization of the blocking reagents (in this case anti-rabbit antibodies) has stopped the migration of the rabbit-HRP antibodies to the lower layers. Without the blocking layer, HRP linked to the antibodies migrated to the layer with immobilized substrate and produced light. Light signal recorded in the presence of blocking pad was 5-times lower than without blocking pad. This may be due to the leaking of the blocker to the test zone. Addition of another blocking layer and better pads separation will solve this issue. The next step is to determine the effect of sample addition. Increasing HRP concentration inside the solution will induce greater luminescent respond. Indeed. Higher solution volumes provided stronger light responses (FIG. 6).

### Example 7: Optimization of the Immobilization Procedures

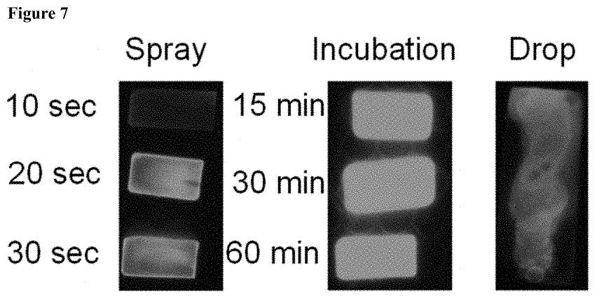

FIG. 7 shows different immobilization approaches used for determining the optimum fixing procedure. For all the experimental evaluations, 1:2000 dilution of antibodies conjugated to HRP were used. In the first approach, antibodies were dispensed on the membrane by chromatographic spread at different durations (FIG. 7, left). In the second approach, membranes were incubated in 10 mL of antibodies solution, and in the last approach, antibody solutions were slowly and carefully drop-deposited in the middle of the nitrocellulose sheet. All membranes were dried in a closed chamber at room temperature. A high-uniformity blocking layer was achieved by incubating membranes within antibodies solution, and all tested incubation durations produced a similar uniform layer. Membranes treated with a dispenser provided the worst results (FIG. 7, middle). In this case, the treatment time influenced the immobilization efficiency, where longer exposure produces higher uniformity. However, even in the case of the highest tested time, the sprayed membrane demonstrated lower immobilization efficiency, when compared to the incubation approach. Finally, antibody solutions from which drops were deposited at the center of the membrane were shown to provide the worst results. During the drying phase, antibodies migrated from the drop site to the membrane borders (FIG. 7, right). Indeed, the main purpose of the blocking layer is to prevent nontarget analyte-bound antibodies-HRP to move to the next upper levels of the assay, thus enabling one to eliminate false positive responses. Thus, uniformity of the surface is crucial, and therefore the incubation approach was adapted for all subsequent experiments.

### Example 8: Determination of Leaching of the Antibodies from the Nitrocellulose Membrane

Another important issue in this important anti-analyte immobilization layer is the possibility that fixed cells may leach away from the nitrocellulose layer during operation of the bioassay. This produces false responses and, thus, should be prevented. FIG. 8 shows the possible leaching of the cells from membrane. There were light responses in the absorption membrane (first) after the first blocking layer, suggesting that diffusion of unbound cells or antibodies from blocking membrane to the next layer had taken place. For the next two absorption membranes (second and third), no visible light response was recorded, suggesting strong cell linkage to the nitrocellulose membrane. Nevertheless, the light response from the first absorption membrane is still more than 100-fold lower than the blocking layer responses. The results suggest that only a very few bacteria/antibodies or antibodies complexes were unbound during the immobilization processes. In terms of the long-term stability of the dried capture E. coli layer and its antigenic stability over time, it is shown that the epitopes remain intact during at least a week; however, long-term studies have not been conducted so far, one of the reasons being the new scenario of future pad design that will have cross-linked immobilized bacteria directly on the membrane which will then be freeze-dried to ensure long-term storage and stability of the antigenic constituents of the bacterium. In addition, the data show that, several days after preparing the stacks, an immunoassay can be successfully done, thus showing a proof of principle that all reagents and immune-reagents used can be stable at least over a short period.

### Example 9: Correlation Curve

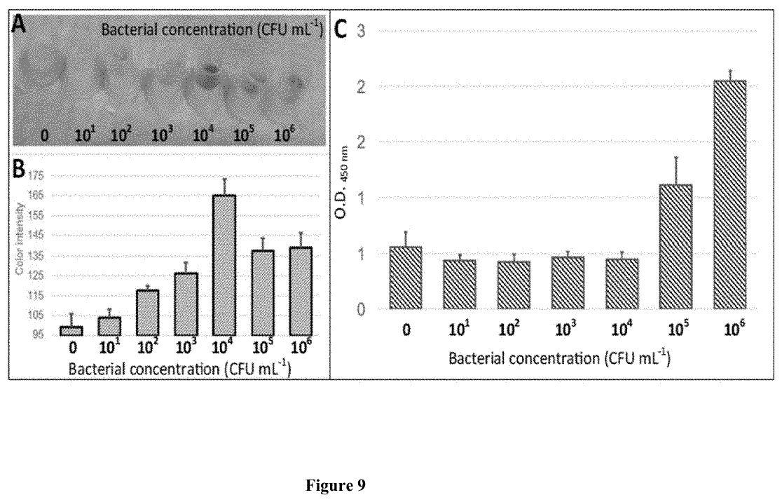

The assay sensitivity of the device of the disclosure was compared to that of colorimetric ELISA, the accepted standard in pathogen detection. Prior to bacterial measurement, the ELISA protocol was optimized in order to determine the optimum antibody volume and concentration for E. coli detection. The stacks immunoassay threshold sensitivity (1×10² cfu/mL) (FIGS. 9A and B) was higher than that of ELISA (1×10⁵ cfu/mL) (FIG. 9C). FIG. 9B shows a clear dose dependence of the stack response, where increasing the bacterial concentration induced an increase in color intensity on the pad surface. Typical ELISA sensitivity to the bacterial pathogens lies between 10⁵ and 10⁷ cfu mL⁻¹, which is 1,000 fold higher than the present approach and may be inadequate for the detection of pathogens in some cases. Alternative ELISA approaches (e.g., methods based on addition different nanocomposites (LOD=60 cfu mL⁻¹), low-fouling surface plasmon resonance (LOD=<50 cfu mL⁻¹), gold (LOD=10 cfu mL⁻¹) or immune-magnetic/gold nanoparticles (LOD=68 cfu mL⁻¹) and carbon nanotubes (LOD=100 cfu mL⁻¹) have shown similar or better sensitivity. However, all these approaches are much more complicated, time consuming and require trained lab skills and equipment. PCR based approaches are more sensitive, but not for the field as of yet.

### Example 10: Comparison of StackPad and ELISA Assay

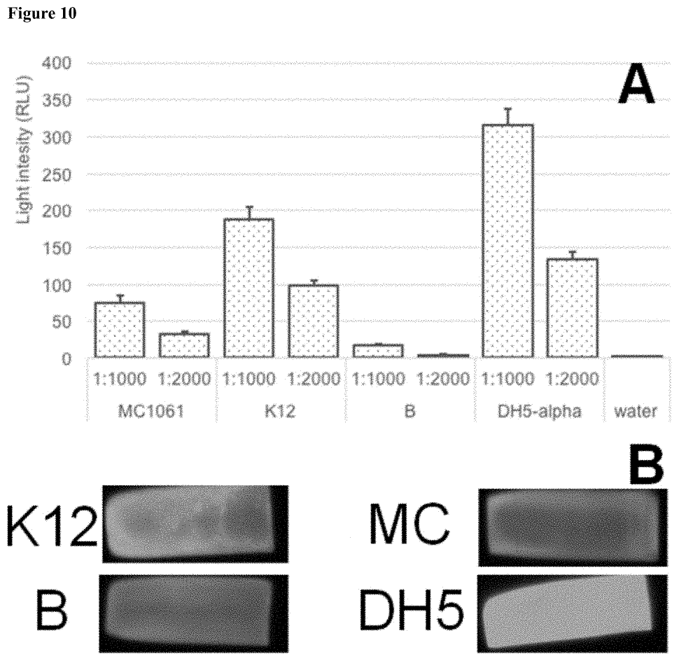

The next important optimization step that was tested involved the specificity of the antibodies to the target bacterial strains. Four different E. coli strains were used, while the chosen capture antibodies were elicited against the DH5α strain. Both of the used technologies [e.g., ELISA (FIG. 10A) and nitrocellulose membrane (FIG. 10B)] have shown a similar behavior with the tested microorganisms. A higher response was obtained for DH5α and its derivative K12 strains. E. coli type B had shown the lowest cross-reactive response, suggesting that the antibodies used were useful. In both techniques the tested E. coli strains showed varying degrees of cross-specificity (DH5α>K12 >MC1061 >B) and that the immobilization procedure on the nitrocellulose membrane is useful as the blocking layer.

### Example 11: Effect of the Blocking Layer on the Stack Pad Assay Functionality

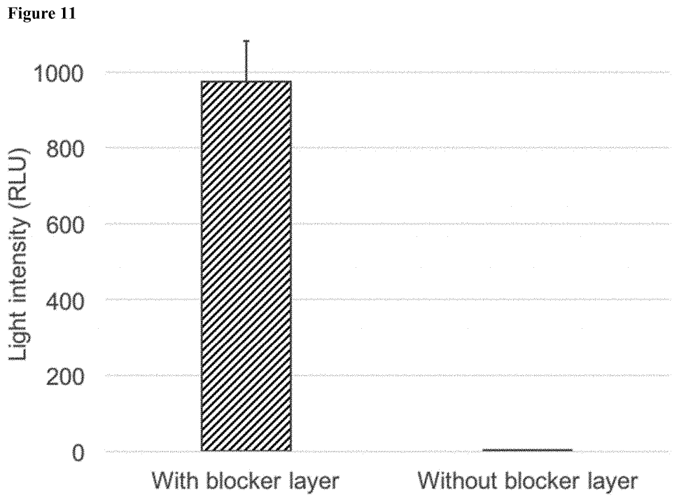

In the next step the entire immunoassay setup was evaluated to confirm the capability of the blocking layer to prevent non-captured HRP-labeled antibodies from flowing through to the final membrane step. This was tested with two different samples, one positive with DH5α strain cells at a concentration of 10⁶ cell mill and a negative control consisting of uncontaminated water. FIG. 11 shows that the setup exposed to the negative control water sample had a 1000-fold lower response than with samples contaminated (spiked) with bacteria, thus confirming the important role of the blocking layer, where unbound antibodies are not able to bypass the immobilized bacteria in the nitrocellulose (blocking) layer. In the case of bacteria in positive controls that will conjugate to the HRP-labeled antibodies and will migrate as a complex through the blocking layer unchallenged so as to migrate up to the substrate (upper) layer. The low background light values (in the negative control) suggested that few antibodies diffused through the blocking layer. If unbound antibodies or bacteria/antibodies complexes leach out of the nitrocellulose membrane, then a false positive would be obtained, but these are not observed (FIG. 8).

### Example 12: How the Number of Blocking Layers and Antibody Concentration Affect the Immunoassay Efficiency

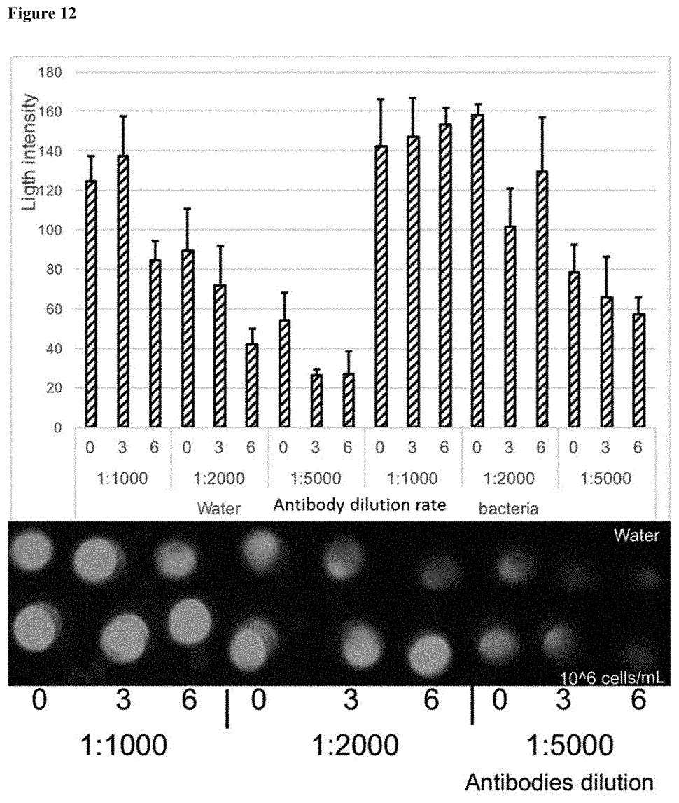

The sensitivity of an immunoassay is usually determined by its efficiency in differentiating the signal output from the background values. The next step was to reduce the background light levels, by adding more blocking layers and optimizing the antibody concentrations. Three different conformations were tested, with zero, three, and six blocking layers. For each, three different anti-E. coli—HRP antibody concentrations (1:1000, 1:2000, and 1:5000) were evaluated (FIG. 12). Each setup was exposed to uncontaminated water as the negative control and bacterial DH5α strain cells (10⁶ cell mL⁻¹) as the positive control. In general, these results may be separated into two parts, high (1:1000) or low (1:2000 and 1:5000) labeled antibody concentrations. For 1:1000 antibody dilution, the experimental test setup response was not linear in some cases. Nevertheless, the output values in the negative controls were lower than the positive, and no significant difference was recorded between the different conformations exhibiting different numbers of blocking layers. A possible reason for this phenomenon may be due to the relatively high antibody concentrations. Oversaturation of antibodies resulted in an insufficient overall capture by E. coli of all the available antibodies; thus, a number of them ended up passing to the upper layers and produce false responses. Indeed, increasing the number of blocking layers decreased the signal values in water samples. Nevertheless, in all tested conformations, the signal output values of tests exposed to bacteria were higher than those of non-spiked water. In the case of lower antibody concentrations (e.g., 1:2000 and 1:5000) the blocking layers reduced non-conjugated antibody flow, while reducing false responses in the system. One must realize that there is a direct correlation between antibody concentrations and the blocking efficiency of the system. At the lower tested concentration, the need for additional numbers of blocking layers was reduced. The optimal configuration between positive and negative controls was shown to be the presence of six blocking layers with a membrane modified by exposure to an HRP-antibody dilution at 1:2000.

### Example 13: Correlation Curve of the ELISA and Stack Pad Assay to the Different DH5α Concentrations

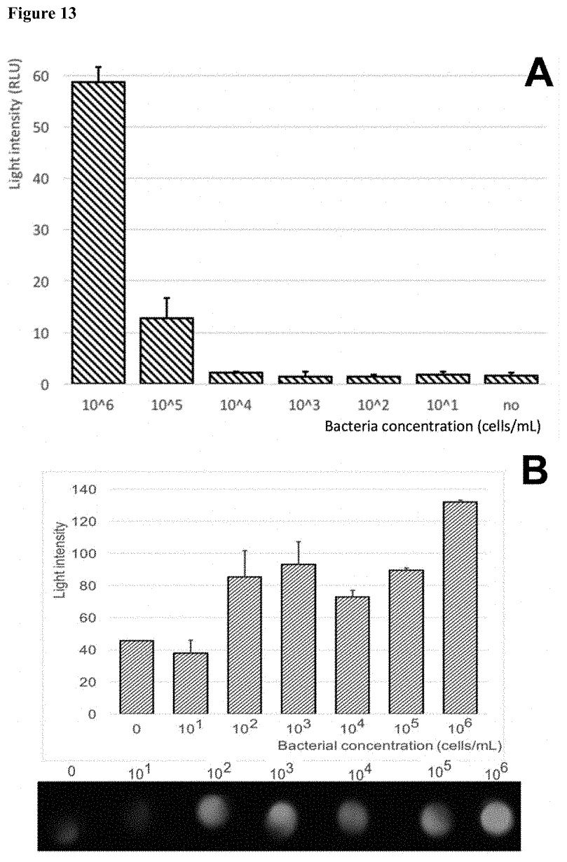

ELISA is commonly used in determining antibacterial antibodies or pathogen antigens but has a relatively poor sensitivity when measuring whole-cell microorganisms. Infectious doses of E. coli (10 cfu mL⁻¹), Salmonella, Listeria (1×10³ cfu mL⁻¹), and Campylobacter (1×10² cfu mL⁻¹) are much lower than the detection limits of most known prior art ELISA immunoassays (1×10⁴-10⁶ cfu mL⁻¹). As seen in FIG. 13, the threshold sensitivity of the immunoassay stack pads was 1×10² cfu mill, about 100 times lower than ELISA performance. Nevertheless, the sensitivity of the present test is still 10-folds higher than the lowest known infectious dose of pathogenic E. coli, albeit sufficiently sensitive to test for other infectious microorganisms (e.g., Salmonella, Listeria, Campylobacter). Furthermore, an interesting fact has higher, or similar, sensitivity to otherwise usually accepted as exhibiting greater sensitivity biosensors including a high-density microelectrode array (10⁴ cfu mill), quartz crystal microbalance immunosensors (10⁵ cfu SPR (surface plasmon resonance) (10⁶ cfu and acoustic wave immunosensor (10⁶ cfu mL⁻¹) known from the prior art. Similar sensitivity was achieved with magnetic nanoparticle clusters and optical nanoparticle probes and higher sensitivity with PCR procedures. However, the aforementioned technologies remain laboratory-based, requiring dedicated personnel and instrumentation.

### Example 14: Determining the Specificity of the Setup

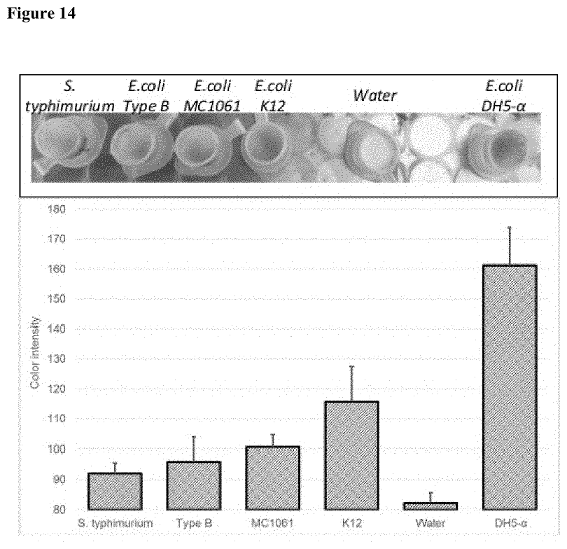

The specificity of the stacked pads immunoassay was determined by comparing five different bacterial strains (e.g., four E. coli and one Salmonella Typhimurium) (FIG. 14). All E. coli strains, besides the target DH5α cells, exhibited much lower color changes, confirming the usefulness of the detecting immunoglobulins used. Lower signal generation of the non-target E. coli strains may be explained by the reduced common number of epitopes shared by the microorganisms. Salmonella Typhimurium, a food pathogen, gave the lowest color change. It is expected that some cross-reaction will occur, as both E. coli and Salmonella Typhimurium strains belong to the Enterobactericeae enteric group and consequently are known to share some molecular similarity in their O or K antigens, which are part of the target structures in the used antibodies. Such cross reactions are known in the art.

### Example 15: Response of the StackPad System to Different Environmental Water

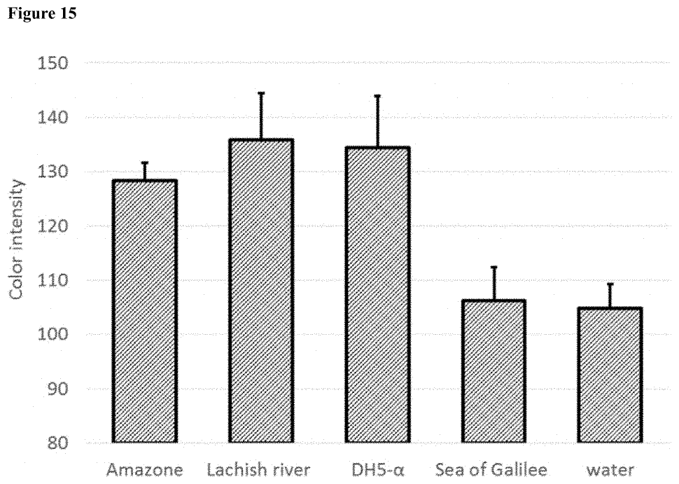

Three different environmental water sources were tested (FIG. 15). The first water was from Lachish River, the second was collected from the Amazon River and the third one from the Sea of Galilee. For both river samples, there were color changes, suggesting the presence of related bacteria in the water (FIG. 15). Indeed, overnight incubation on LB agar plates has shown the presence of similar bacteria in the water. A positive response with the stack pad system indicates that E. coli cells are present. Nevertheless, even though the strength of the detection values of the positive responses was similar to the DH5-α results (FIGS. 9 and 14), it cannot ascertain that the strain is such as it may be due herein documented cross-reactivity. At the present stage only the presence of the microorganism genus in the sample was confirmed, while further optimization of the antibodies specificity will provide more realistic results. Stack pads exposed to the Sea of Galilee water samples have shown similar values to those of clear water. Thus, negative (Sea of Galilee) and positive (Lachish and Amazone rivers) detection results have clearly shown the capability of the system of the present disclosure as an alert system and there is indication of a semi-quantitation potential.

### Example 16: Response of the Flow Pad Biosensor Based on Electrochemical Approaches

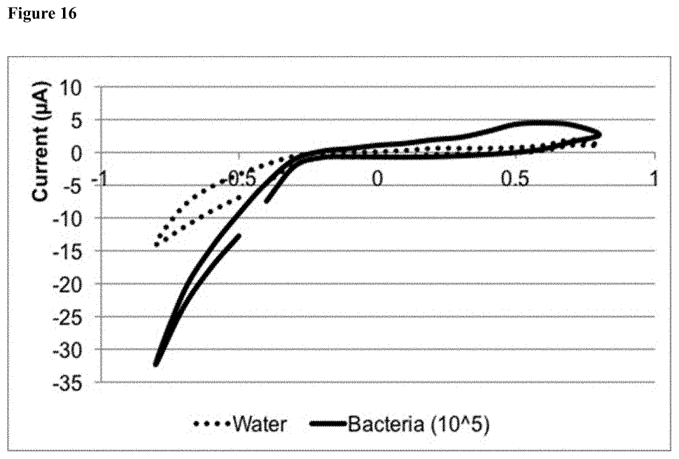

Pads of the present disclosure were placed above screen-printed electrodes (BioAnalytics). FIG. 16 shows the recorded cyclic voltammetry of the setup, the solide CV represents the response of the setup to cleat water, while the dashed CV represents the response to the 10⁵ cells/mL E. coli in the sample.

### Example 17: Incorporation of a Stopping Layer

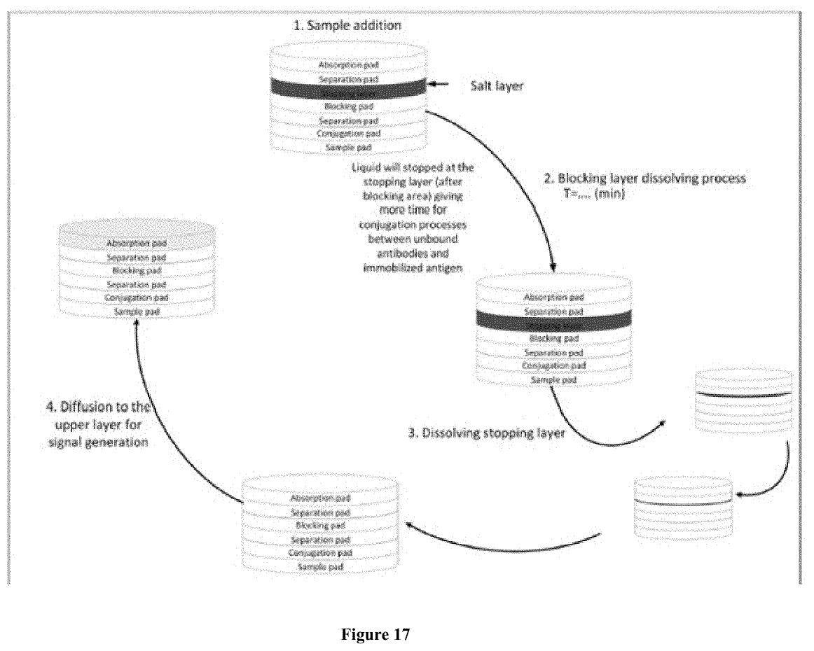

In order to increase the conjugation time between unbound antibodies a stopping layer (salt or some polymer with time depended dissolving parameters) may be placed between blocking layers and upper absorption pad (FIG. 17). A sample will flow through setup to the blocking layer and will stop there. Liquid will propagate to the upper layer only after dissolving this barrier. This will increase the reaction time between unbound antibodies and nitrocellulose immobilized antigen.

### Example 18: Fiberglass Paper as a Blocking Layer

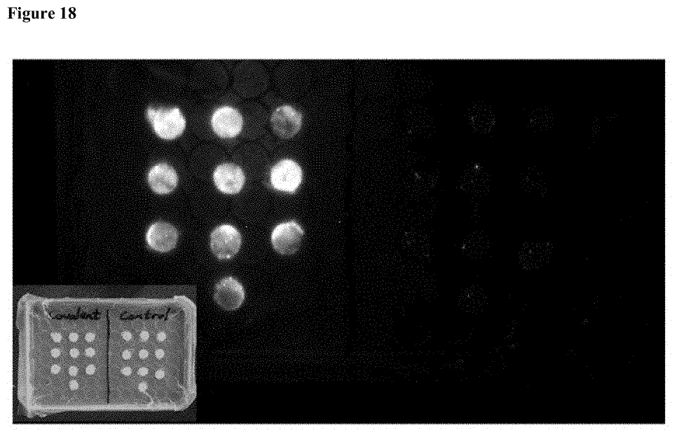

FIG. 18 demonstrates the capability of covalent binding of biological molecules above fiberglass paper. These modified layers will be used in the stackpads setup as blocking layers.

### Example 19: Polyvinylidene Difluoride (PVDF) as Blocking Membrane

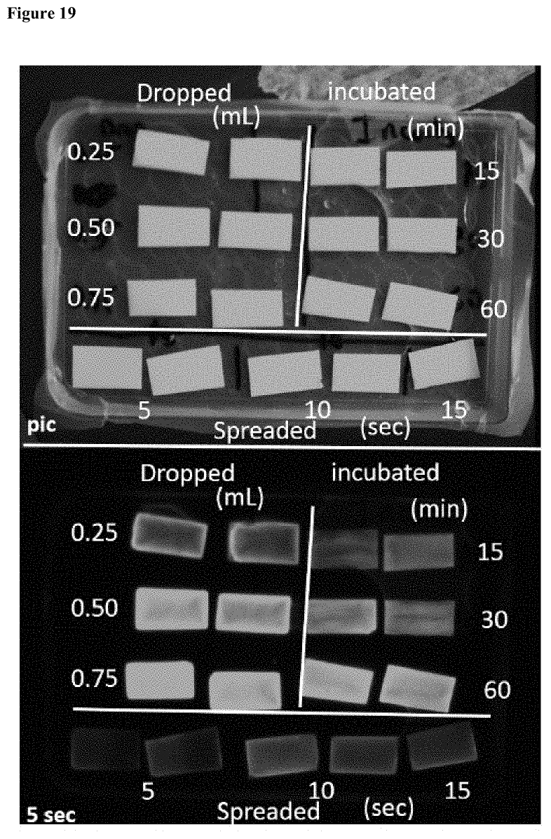

FIG. 19 demonstrates that biological molecules (antigens) may be immobilized above PVDF. Membranes were exposed to the different fixing approaches. Then washed with PBST and exposed to the HRP conjugated antibodies for one hour. After additional washing step substrate (luminol:H₂O₂) was placed above for signal generation. During washing steps all unbound biological materials were washed out and only the ones fixed on the membrane will generate light in the end. These results suggesting that efficiency of the immobilization procedure is depend on immobilization procedure, while, in this case the most efficient was placing and drying antigen solution above membranes. But it is clear that there were immobilized molecules on the surface that were connected to the antibodies. Similar to the nitrocellulose, PVDF membranes may be used in the future stackpads formation as blocking layer.

### Example 20: Determination of Neisseria gonorrhoeae and Dengue Virus

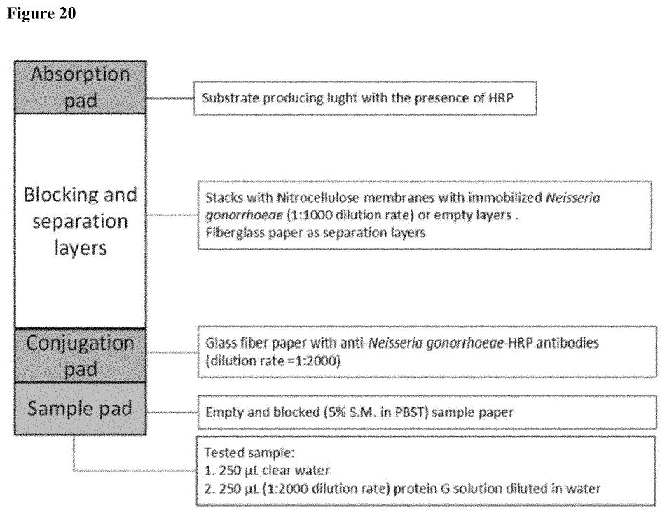

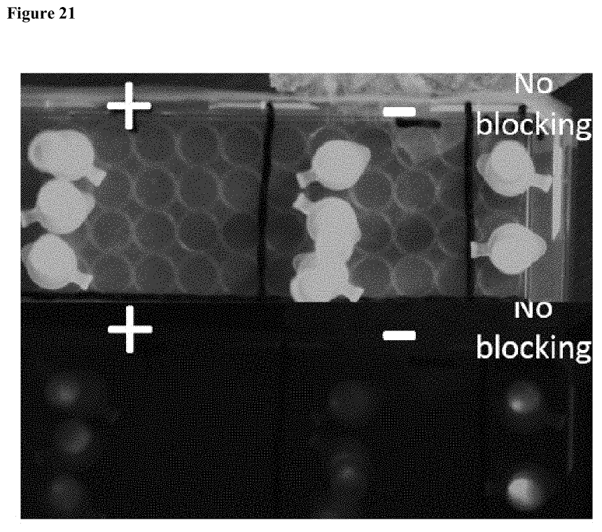

StackPads setup was used for determination sexual transmitted diseases. FIG. 20 shows the setup structure and materials types and concentrations. FIG. 21 demonstrates the capability of the device of the present disclosure to sense the presence of Neisseria gonorrhoeae in the water samples. The highest signals were observed with the setups without active blocking layers, suggesting uncontrolled antibodies flow to the upper layers. Setups exposed to the positive samples (with gonorrhea) showed much higher responses than stackpads exposed to the clear water.

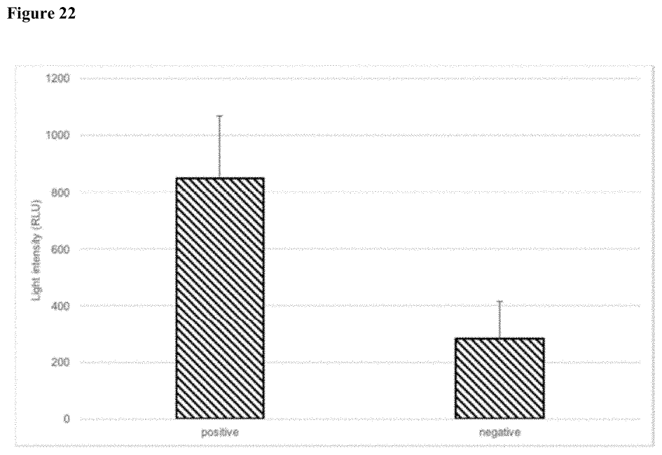

As in previous cases FIG. 22 demonstrates the capability of the present device to sense the presence of Dengue virus in the water samples. Setups exposed to the positive samples (with Dengue virus) showed much higher responses than stackpads exposed to the clear water.

The disclosure has been described broadly and generically herein. Each of the narrower species and subgeneric groupings falling within the generic disclosure also form part of the disclosure. This includes the generic description of the disclosure with a proviso or negative limitation removing any subject-matter from the genus, regardless of whether or not the excised material is specifically recited herein. Other aspects are within the following claims. In addition, where features or aspects of the disclosure are described in terms of Markush groups, those skilled in the art will recognize that the disclosure is also thereby described in terms of any individual member or subgroup of members of the Markush group.

One skilled in the art would readily appreciate that the present disclosure is well adapted to carry out the objects and obtain the ends and advantages mentioned, as well as those inherent therein. Further, it will be readily apparent to one skilled in the art that varying substitutions and modifications may be made to the disclosure disclosed herein without departing from the scope and spirit of the disclosure. The compositions, methods, procedures, treatments, molecules and specific compounds described herein are presently representative of some aspects are exemplary and are not intended as limitations on the scope of the disclosure. Changes therein and other uses will occur to those skilled in the art which are encompassed within the spirit of the disclosure are defined by the scope of the claims. The listing or discussion of a previously published document in this specification should not necessarily be taken as an acknowledgement that the document is part of the state of the art or is common general knowledge.

The disclosure illustratively described herein may suitably be practiced in the absence of any element or elements, limitation or limitations, not specifically disclosed herein. Thus, for example, the terms “comprising”, “including”, “containing”, etc. shall be read expansively and without limitation. The word “comprise” or variations such as “comprises” or “comprising” will accordingly be understood to imply the inclusion of a stated integer or groups of integers but not the exclusion of any other integer or group of integers. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed. Thus, it should be understood that although the present disclosure has been specifically disclosed by exemplary aspects and optional features, modification and variation of the disclosures embodied therein herein disclosed may be resorted to by those skilled in the art, and that such modifications and variations are considered to be within the scope of this disclosure.

The content of all documents and patent documents cited herein is incorporated by reference in their entirety.

1. A device to determine or quantify the presence of an analyte molecule, virus, or cell of interest in a sample, wherein said device comprises at least one unit of stacked layers comprising:
at least one blocking layer comprising a first membrane and a plurality of the analyte molecules, virus, or cells of interest attached to the membrane.

2. The device according to claim 1, wherein the unit of stacked layers further comprises:
a sample layer comprising a second membrane;
at least one conjugation layer, each conjugation layer comprising a respective third membrane and a first binding molecule to bind to the analyte molecule, virus, or cells of interest, wherein:
the first binding molecule is conjugated to a reporter molecule, or
a second conjugation layer comprising a respective third membrane and a second binding molecule binding to the first binding molecule, wherein the second binding molecule is conjugated to the reporter molecule; and
an absorption layer comprising a fourth membrane.

3. The device according to claim 2, wherein the layer composition of the unit of stacked layers is:
the sample layer; the at least one conjugation layer; the at least one blocking layer; and the absorption layer, wherein a flow direction of the sample is from the sample layer to the absorption layer.

4. The device according to claim 2, wherein each of the sample layer, the at least one conjugation layer, the at least one blocking layer, and the absorption layer are separated by a separation layer comprising a separation membrane.

5. The device according to claim 1, wherein the unit of stacked layers further comprises a stop layer that is, in the flow direction, immediately located behind the at least one blocking layer, wherein the stop layer is dissolved upon contact with the sample.

6. The device according to claim 5, wherein the stop layer comprises a salt or polymer.

7. The device according to claim 1, wherein the sample is a liquid sample.

8. The device according to claim 2, wherein the respective third membrane of the at least one conjugation layer and the first binding molecule are non-covalently attached to each other.

9. The device according to claim 1, wherein the plurality of analyte molecules, virus, or cells of interest are covalently immobilized on the first membrane of the at least one blocking layer.

10. The device according to claim 2, wherein the absorption layer further comprises a substrate for the reporter molecule.

11. The device according to claim 2, wherein the first and the second binding molecule are independently selected from the group consisting of a protein, a nucleotide, an aptamer, a natural ligand, and any combinations thereof.

12. The device according to claim 2, wherein the reporter molecule is selected from the group consisting of a protein, a nucleotide, a dye, a redox molecule, gold, silver, platinum, and any combinations thereof.

13. The device according to claim 2, wherein the membranes of the sample layer, the at least one conjugation layer, the at least one blocking layer, and the absorption layer are independently selected from the group consisting of cellulose acetate membrane, nitrocellulose membrane, cellulose ester membrane, polysulfone (PS) membrane, polyether sulfone (PES) membrane, polyacrilonitrile (PAN) membrane, polyamide membrane, polyimide membrane, polyethylene and polypropylene (PE and PP) membrane, polytetrafluoroethylene (PTFE) membrane, polyvinylidene fluoride (PVDF) membrane, polyvinylchloride (PVC) membrane, fiberglass paper membrane, and any combinations thereof.

14. The device according to claim 1, wherein the unit of stacked layers comprises at least 6 blocking layers.

15. The device according to claim 1, wherein the analyte molecule, virus, or cell of interest is selected from the group consisting of a cell, a protein, a virus, a viral toxin, a bacterial toxin, a biotoxin, parasite, fungus, a nucleotide, a natural ligand, and any combinations thereof.

16. The device according to claim 1, wherein the device comprises at least three units of stacked layers.

17. A method to prepare a device to determine or quantify the presence of an analyte molecule, virus, or cell of interest in a sample, wherein said device comprises at least one unit of stacked layers comprising at least one blocking layer comprising a first membrane and a plurality of the analyte molecules, virus, or cells of interest attached to the membrane; and at least one conjugation layer, each conjugation layer comprising a respective third membrane and a first binding molecule to bind to the analyte molecule, virus, or cells of interest, the method comprising:
incubating and drying the respective third membrane of the at least one conjugation layer and a sample comprising the first binding molecule; and
immobilizing the plurality of analyte molecules, virus, or cells of interest on the first membrane of the at least one blocking layer.

18. (canceled)

19. (canceled)

20. A method to determine or quantify the presence of an analyte molecule, virus, or cell of interest in a sample, the method comprising:
contacting the sample and a device comprising at least one unit of stacked layers comprising at least one blocking layer comprising a first membrane and a plurality of the analyte molecules, virus, or cells of interest attached to the first membrane; a sample layer comprising a second membrane; and at least one conjugation layer, each conjugation layer comprising a respective third membrane and a binding molecule to bind to the analyte molecule, virus, or cells of interest, wherein the binding molecule to conjugated to a reporter molecule; and
determining or quantifying the presence of the analyte molecule, virus, or cell of interest by detecting a reporter molecule dependent signal.

21. (canceled)

22. (canceled)

23. (canceled)

24. (canceled)

25. The method according to claim 20, wherein the method determines or quantifies the analyte molecule, or cell of interest in the sample that is indicative for:
influenza AB infection, respiratory syncytial virus (RSV) infection, parainfluenza infection, adenovirus infection, and/or metapneumovirus infection;
dengue virus infection, Zika virus infection, malaria infection, lassa virus infection, ebola virus infection, west-nile virus infection, and/or yellow fever virus infection;
fertility; or
diarrhea, travel fever, child fever, meningitis/encephalitis, respiratory diseases, sepsis, hemorhhagi fever and/or cancer.
